(19) United States
(12) Patent Application Publication
(10) Pub. No.: US 2022/0193050 A1
(43) Pub. Date: Jun. 23, 2022

(54) ORAL FORMULATION FOR A PD-L1 INHIBITOR

(71) Applicant: Incyte Corporation, Wilmington, DE (US)

(72) Inventors: Yonglai Yang, Hockessin, DE (US); Trupti Sheth, Newark, DE (US); Xinsheng Xie, Exton, PA (US)

(21) Appl. No.: 17/554,340

(22) Filed: Dec. 17, 2021

Related U.S. Application Data
(60) Provisional application No. 63/127,726, filed on Dec. 18, 2020.

(51) Int. Cl. A61K 31/4375; A61K 9/00; A61K 47/12

(52) U.S. Cl. CPC A61K 31/4375; A61K 47/12; A61K 9/0053

(57) ABSTRACT

Disclosed are oral formulations for a compound which modulates PD-1/PD-L1 protein/protein interaction, or a pharmaceutically acceptable salt thereof, which do not require cold chain storage.

### CROSS-REFERENCE TO RELATED APPLICATIONS

The present application claims the benefit of U.S. Provisional Application No. 63/127,726, filed Dec. 18, 2020, which is incorporated herein by reference in its entirety.

### FIELD OF THE INVENTION

The present application is directed to stable oral formulations for a compound which modulates PD-1/PD-L1 protein/protein interaction, or a pharmaceutically acceptable salt thereof.

### BACKGROUND OF THE INVENTION

The immune system plays an important role in controlling and eradicating diseases such as cancer. However, cancer cells often develop strategies to evade or to suppress the immune system in order to favor their growth. One such mechanism is altering the expression of co-stimulatory and co-inhibitory molecules expressed on immune cells (Postow et al, J. Clinical Oncology 2015, 1-9). Blocking the signaling of an inhibitory immune checkpoint, such as PD-1, has proven to be a promising and effective treatment modality.

Programmed cell death-1 (PD-1), also known as CD279, is a cell surface receptor expressed on activated T cells, natural killer T cells, B cells, and macrophages (Greenwald et al, Annu. Rev. Immunol 2005, 23:515-548; Okazaki and Honjo, Trends Immunol 2006, (4):195-201). It functions as an intrinsic negative feedback system to prevent the activation of T-cells, which in turn reduces autoimmunity and promotes self-tolerance. In addition, PD-1 is also known to play a critical role in the suppression of antigen-specific T cell response in diseases like cancer and viral infection (Sharpe et al, Nat Immunol 2007 8, 239-245; Postow et al, J. Clinical Oncol 2015, 1-9).

The structure of PD-1 consists of an extracellular immunoglobulin variable-like domain followed by a transmembrane region and an intracellular domain (Parry et al, Mol Cell Biol 2005, 9543-9553). The intracellular domain contains two phosphorylation sites located in an immunoreceptor tyrosine-based inhibitory motif and an immunoreceptor tyrosine-based switch motif, which suggests that PD-1 negatively regulates T cell receptor-mediated signals. PD-1 has two ligands, PD-L1 and PD-L2 (Parry et al, Mol Cell Biol 2005, 9543-9553; Latchman et al, Nat Immunol 2001, 2, 261-268), and they differ in their expression patterns. PD-L1 protein is upregulated on macrophages and dendritic cells in response to lipopolysaccharide and GM-CSF treatment, and on T cells and B cells upon T cell receptor and B cell receptor signaling. PD-L1 is also highly expressed on almost all tumor cells, and the expression is further increased after IFN-γ treatment (Iwai et al, PNAS 2002, 99(19):12293-7; Blank et al, Cancer Res 2004, 64(3):1140-5). In fact, tumor PD-L1 expression status has been shown to be prognostic in multiple tumor types (Wang et al, Eur J Surg Oncol 2015; Huang et al, Oncol Rep 2015; Sabatier et al, Oncotarget 2015, 6(7): 5449-5464). PD-L2 expression, in contrast, is more restricted and is expressed mainly by dendritic cells (Nakae et al, J Immunol 2006, 177:566-73). Ligation of PD-1 with its ligands PD-L1 and PD-L2 on T cells delivers a signal that inhibits IL-2 and IFN-γ production, as well as cell proliferation induced upon T cell receptor activation (Carter et al, Eur J Immunol 2002, 32(3):634-43; Freeman et al, J Exp Med 2000, 192(7):1027-34). The mechanism involves recruitment of SHP-2 or SHP-1 phosphatases to inhibit T cell receptor signaling such as Syk and Lck phosphorylation (Sharpe et al, Nat Immunol 2007, 8, 239-245). Activation of the PD-1 signaling axis also attenuates PKC-θ activation loop phosphorylation, which is necessary for the activation of NF-κB and AP1 pathways, and for cytokine production such as IL-2, IFN-γ and TNF (Sharpe et al, Nat Immunol 2007, 8, 239-245; Carter et al, Eur J Immunol 2002, 32(3):634-43; Freeman et al, J Exp Med 2000, 192(7):1027-34).

Several lines of evidence from preclinical animal studies indicate that PD-1 and its ligands negatively regulate immune responses. PD-1-deficient mice have been shown to develop lupus-like glomerulonephritis and dilated cardiomyopathy (Nishimura et al, Immunity 1999, 11:141-151; Nishimura et al, Science 2001, 291:319-322). Using an LCMV model of chronic infection, it has been shown that PD-1/PD-L1 interaction inhibits activation, expansion and acquisition of effector functions of virus-specific CD8 T cells (Barber et al, Nature 2006, 439, 682-7). Together, these data support the development of a therapeutic approach to block the PD-1-mediated inhibitory signaling cascade in order to augment or “rescue” T cell response.

Accordingly, there is a need for new formulations of compounds that block PD-1/PD-L1 protein/protein interaction. This disclosure addresses this need and others.

### SUMMARY

The present disclosure provides, inter alia, pharmaceutical formulations, comprising a compound of Formula (I):

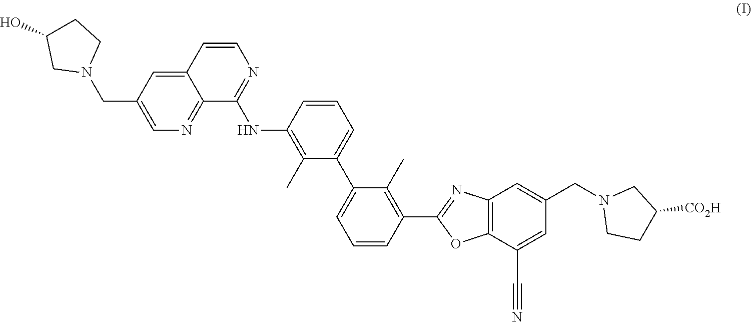 [SMILES: Cc1c(Nc2nccc3cc(CN4CC[C@@H](O)C4)cnc23)cccc1-c1cccc(-c2nc3cc(CN4CC[C@@H](C(=O)O)C4)cc(C#N)c3o2)c1C]

or a pharmaceutically acceptable salt thereof, and a stabilizing agent.

The present disclosure further provides oral dosage forms comprising the pharmaceutical formulations disclosed herein.

The present disclosure further provides methods of reducing the amount of cell surface PD-L1, said methods comprising administering to a patient in need thereof an oral dosage form disclosed herein.

The present disclosure further provides methods of decreasing or reducing the interaction of PD-1 and PD-L1, said methods comprising administering to a patient in need thereof an oral dosage form disclosed herein.

The present disclosure further provides methods of enhancing, stimulating and/or increasing an immune response in a patient in need thereof, said methods comprising administering to the patient in need thereof an oral dosage form disclosed herein.

The present disclosure further provides methods of treating a PD-1-related disease or condition, said methods comprising administering to a patient in need thereof an oral dosage form disclosed herein.

### DETAILED DESCRIPTION

Compound 1 ((R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid) having Formula (I) below is a potent PD-L1 inhibitor. Compound 1 can bind to cell surface PD-L1 and induce PD-L1 internalization, thereby reducing the inhibitory signaling that results from the PD-1-PD-L1 interaction. By reducing PD-1 inhibitory signaling, Compound 1 can increase an immune response and can, therefore, can be used to treat a PD-1-related disease or condition such as cancer.

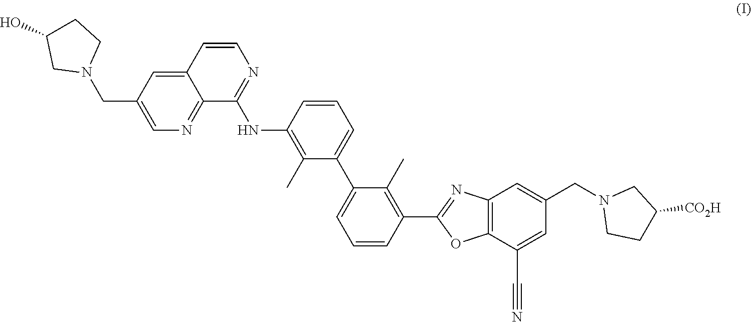 [SMILES: Cc1c(Nc2nccc3cc(CN4CC[C@@H](O)C4)cnc23)cccc1-c1cccc(-c2nc3cc(CN4CC[C@@H](C(=O)O)C4)cc(C#N)c3o2)c1C]

Because Compound 1 is moisture, light, and heat-sensitive, the compound must be stored at freezer temperatures (e.g., −20° C.). Further, pharmaceutical products containing Compound 1 are typically stored at refrigerator temperatures of 2-8° C. As such, there is a need for pharmaceutical formulations and dosage forms of Compound 1 that provide improved stability, more particularly pharmaceutical formulations and dosage forms of Compound I which eliminate the need for cold chain storage. Additionally, it can be challenging to develop dosage forms that contain more than 50% by weight of the drug in the dosage form. This disclosure addresses these needs and others.

Accordingly, it has been discovered that use of a stabilizing agent (such as an organic acid) can unexpectedly provide formulations having stability at a temperature of 25° C. and 60% humidity that is comparable to the stability of formulations without the stabilizing agent at refrigerator storage conditions (e.g., at a temperature of about 2 to about 8° C.). The formulation can also include a diluent having a low moisture content (e.g., having moisture content of not more than 2% or not more than 1.5%) and the dosage forms of the disclosure can be coated in order provide a further barrier against moisture. Further, this stabilized formulation is much more stable than a formulation without a stabilizing agent after 6 months at a temperature of 25° C. and 60% humidity.

### I. Formulations, Dosage Forms and Administration

The present disclosure provides, inter alia, a pharmaceutical formulation, comprising a compound of Formula (I):

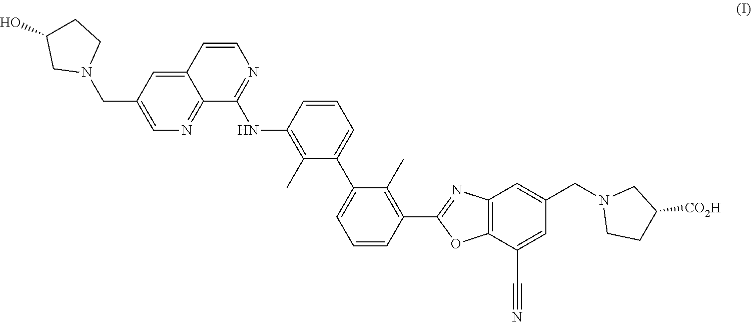 [SMILES: Cc1c(Nc2nccc3cc(CN4CC[C@@H](O)C4)cnc23)cccc1-c1cccc(-c2nc3cc(CN4CC[C@@H](C(=O)O)C4)cc(C#N)c3o2)c1C]

or a pharmaceutically acceptable salt thereof, and a stabilizing agent.

In some embodiments, the compound of Formula (I), or the pharmaceutically acceptable salt thereof, is present as a free base.

In some embodiments, the compound of Formula (I), or the pharmaceutically acceptable salt thereof, is present in an amount on a free base basis of from about 30% to about 80% by weight of the formulation. In some embodiments, the compound of Formula (I), or the pharmaceutically acceptable salt thereof, is present in an amount on a free base basis of from about 40% to about 80% by weight of the formulation. In some embodiments, the compound of Formula (I), or the pharmaceutically acceptable salt thereof, is present in an amount on a free base basis of from about 40% to about 70% by weight of the formulation. In some embodiments, the compound of Formula (I), or the pharmaceutically acceptable salt thereof, is present in an amount on a free base basis of from about 45% to about 70% by weight of the formulation. In some embodiments, the compound of Formula (I), or the pharmaceutically acceptable salt thereof, is present in an amount on a free base basis of from about 45% to about 60% by weight of the formulation. In some embodiments, the compound of Formula (I), or the pharmaceutically acceptable salt thereof, is present in an amount on a free base basis of from about 48% to about 55% by weight of the formulation. In some embodiments, the compound of Formula (I), or the pharmaceutically acceptable salt thereof, is present in an amount on a free base basis of about 53% by weight of the formulation.

In some embodiments, the stabilizing agent is an organic acid or an ester thereof, a metal sulfite, a metal bisulfite, a metal metabisulfite, a metal thiosulfate, a metal formaldehyde sulfoxylate, or an alkylated phenol, or a mixture of any of the aforementioned.

In some embodiments, the stabilizing agent is ascorbic acid, fumaric acid, citric acid, tartaric acid, ascorbyl palmitate, propyl gallate, sodium sulfite, sodium bisulfite, sodium metabisulfite, sodium thiosulfate, sodium formaldehyde sulfoxylate, butylated hydroxyanisole, butylated hydroxytoluene, cysteine, or tocopherol.

In some embodiments, the stabilizing agent is present in an amount of from about 0.001% to about 20% by weight of the formulation. In some embodiments, the stabilizing agent is present in an amount of from about 0.001% to about 10% by weight of the formulation. In some embodiments, the stabilizing agent is present in an amount of from about 0.001% to about 5% by weight of the formulation. In some embodiments, the stabilizing agent is present in an amount of from about 0.001% to about 1% by weight of the formulation. In some embodiments, the stabilizing agent is present in an amount of from about 0.001% to about 0.1% by weight of the formulation. In some embodiments, the stabilizing agent is present in an amount of from about 0.001% to about 0.01% by weight of the formulation. In some embodiments, the stabilizing agent is present in an amount of from about 2% to about 3% by weight of the formulation. In some embodiments, the stabilizing agent is present in an amount of about 2.5% by weight of the formulation.

In some embodiments, the stabilizing agent is an antioxidant. In some embodiments, the antioxidant is ascorbic acid.

In some embodiments, the stabilizing agent is an organic acid. In some embodiments, the organic acid is C₁₋₈ alkyl carboxylic acid, C₂₋₈ alkenyl carboxylic acid, or phenyl carboxylic acid, each of which is optionally substituted by 1, 2, 3, 4, 5, or 6 substituents independently selected from SH, NH₂, OH, and CO₂H. In some embodiments, the organic acid is C₁₋₈ alkyl carboxylic acid, which is optionally substituted by 1, 2, 3, 4, 5, or 6 substituents independently selected from SH, NH₂, OH, and CO₂H. In some embodiments, the organic acid is C₂₋₈ alkenyl carboxylic acid, which is optionally substituted by 1, 2, 3, 4, 5, or 6 substituents independently selected from SH, NH₂, OH, and CO₂H. In some embodiments, the organic acid is fumaric acid, citric acid, succinic acid, adipic acid, maleic acid, sorbic acid, malonic acid, glutaric acid, gluconic acid, lactic acid, glycolic acid, malic acid, tartaric acid, tartronic acid, galactaric acid, glutamic acid, aspartic acid, benzoic acid, phthalic acid, isophthalic acid, terephthalic acid, or trimelitic acid. In some embodiments, the organic acid is fumaric acid, citric acid, succinic acid, adipic acid, maleic acid, sorbic acid, malonic acid, glutaric acid, gluconic acid, lactic acid, glycolic acid, malic acid, tartaric acid, tartronic acid, galactaric acid, glutamic acid, or aspartic acid. In some embodiments, the organic acid is fumaric acid, citric acid, tartaric acid, succinic acid, adipic acid, or maleic acid. In some embodiments, the organic acid is fumaric acid, citric acid, or tartaric acid. In some embodiments, the organic acid is fumaric acid.

In some embodiments, the organic acid is present in an amount of from about 0.5% to about 20% by weight of the formulation. In some embodiments, the organic acid is present in an amount of from about 0.5% to about 10% by weight of the formulation. In some embodiments, the organic acid is present in an amount of from about 0.5% to about 5% by weight of the formulation. In some embodiments, the organic acid is present in an amount of from about 1% to about 10% by weight of the formulation. In some embodiments, the organic acid is present in an amount of from about 1% to about 5% by weight of the formulation. In some embodiments, the organic acid is present in an amount of from about 1% to about 4% by weight of the formulation. In some embodiments, the organic acid is present in an amount of from about 2% to about 3% by weight of the formulation.

In some embodiments, the organic acid is an antioxidant.

In some embodiments, the pharmaceutical formulation provided herein can further include a diluent, a disintegrant, a glidant, a lubricant, a binder, or a combination thereof.

In some embodiments, the pharmaceutical formulation further comprises one or more excipients. In some embodiments, the one or more excipients are present in an amount of from about 5% to about 90% by weight of the formulation. In some embodiments, the one or more excipients are present in an amount of from about 5% to about 70% by weight of the formulation. In some embodiments, the one or more excipients are present in an amount of from about 10% to about 70% by weight of the formulation. In some embodiments, the one or more excipients are present in an amount of from about 10% to about 60% by weight of the formulation. In some embodiments, the one or more excipients are present in an amount of from about 10% to about 50% by weight of the formulation. In some embodiments, the one or more excipients are present in an amount of from about 20% to about 60% by weight of the formulation. In some embodiments, the one or more excipients are present in an amount of from about 20% to about 50% by weight of the formulation.

In some embodiments, the one or more excipients is microcrystalline cellulose, silicified microcrystalline cellulose, mannitol, lactose, sucrose, dextrose, sorbitol, xylitol, starch, sodium starch, calcium phosphate, an alginate, calcium carbonate, sodium carbonate, sodium chloride, calcium sulphate, calcium lactate, sodium chloride, a wax, a clay, talc, tragacanth, glucose, acacia, guar gum, agar, povidone, crospovidone, copovidone, poly(vinyl pyrrolidone-co-vinyl acetate), gelatin, hydroxypropyl cellulose, hydroxypropyl methyl cellulose, hydroxypropyl methylcellulose acetate stearate, methyl cellulose, ethyl cellulose, cellulose, silicified cellulose, carboxymethylcellulose, croscarmellose sodium, carboxymethylcellulose calcium, sodium starch glycolate, an ion-exchange resin, or a mixture of any of the aforementioned.

In some embodiments, calcium lactate is calcium lactate trihydrate.

In some embodiments, calcium sulfate is selected from monobasic calcium sulfate monohydrate or calcium sulfate dihydrate, or a mixture of any of the aforementioned.

In some embodiments, a wax is candelilla wax or carnuba wax, or a mixture of any of the aforementioned.

In some embodiments, a clay is kaolin, bentonite, or magnesium aluminum silicate, or a mixture of any of the aforementioned.

In some embodiments, the lactose is anhydrous lactose, lactose monohydrate, spray-dried monohydrate lactose, or lactose-316 Fast Flo®, or a mixture of any of the aforementioned.

In some embodiments, the cellulose is acidified cellulose, methyl cellulose, ethyl cellulose, or powdered cellulose, or a mixture of any of the aforementioned.

In some embodiments, the starch is corn starch, potato starch, pregelatinized starch, hydrolyzed starch, starch 1500, directly compressed starch, or dry starch, or a mixture of any of the aforementioned.

In some embodiments, the calcium phosphate is dibasic calcium phosphate, dibasic calcium phosphate dehydrate, or tribasic calcium phosphate, or a mixture of any of the aforementioned.

In some embodiments, an alginate is sodium alginate, calcium alginate, potassium alginate, or alginic acid, or a mixture of any of the aforementioned.

In some embodiments, the one or more excipients comprises at least one excipient having a moisture content of not more than about 2% by weight of the excipient. In some embodiments, the one or more excipients comprises at least one excipient having a moisture content of not more than about 1.5% by weight of the excipient. In some embodiments, the one or more excipients, having a moisture content of not more than about 2% or not more than about 1.5% by weight of the excipient, comprises at least 15% by weight of the formulation. In some embodiments, the one or more excipients, having a moisture content of not more than about 2% or not more than about 1.5% by weight of the excipient, comprises at least 20% by weight of the formulation. In some embodiments, the one or more excipients, having a moisture content of not more than about 2% or not more than about 1.5% by weight of the excipient, comprises at least 30% by weight of the formulation. In some embodiments, the one or more excipients, having a moisture content of not more than about 2% or not more than about 1.5% by weight of the excipient, comprises about 35% by weight of the formulation.

In some embodiments, the pharmaceutical formulation further comprises a glidant component or lubricant component.

In some embodiments, the glidant component or lubricant component comprises magnesium stearate, silicon dioxide, sodium stearyl fumarate, calcium stearate, stearic acid, a hydrogenated oil, polyethylene glycol, a starch, an alginate, glyceryl monostearate, glyceryl behenate, glyceryl palmitostearate, a fatty acid, a poloxamer, a metal stearate, a metal fatty acid salt, talc, a clay, or a silicate, or a mixture of any of the aforementioned.

In some embodiments, a fatty acid is a C₆₋₂₀ alkyl carboxylic acid or a C₆₋₂₀ alkenyl carboxylic acid. In some embodiments, the fatty acid is stearic acid, oleic acid, myristic acid, or a mixture of any of the aforementioned.

In some embodiments, a metal fatty acid salt is a salt of a fatty acid where the counter ion is an alkali metal or alkaline earth metal counterion (e.g., Na, K, or Mg). In some embodiments, the metal fatty acid salt is magnesium stearate, sodium stearate, potassium stearate, or a mixture of any of the aforementioned.

In some embodiments, a metal stearate is an alkali metal or alkaline earth metal stearate. In some embodiments, the metal stearate is magnesium stearate, sodium stearate, potassium stearate, or a mixture of any of the aforementioned.

In some embodiments, a metal lauryl sulfate is an alkali metal or alkaline earth metal lauryl sulfate. In some embodiments, the metal lauryl sulfate is magnesium lauryl sulfate, sodium lauryl sulfate, potassium lauryl sulfate, or a mixture of any of the aforementioned.

In some embodiments, the pharmaceutical formulation further comprises a diluent component.

In some embodiments, the diluent component is present in an amount of from about 5% to about 90% by weight of the formulation. In some embodiments, the diluent component is present in an amount of from about 5% to about 70% by weight of the formulation. In some embodiments, the diluent component is present in an amount of from about 10% to about 70% by weight of the formulation. In some embodiments, the diluent component is present in an amount of from about 10% to about 60% by weight of the formulation. In some embodiments, the diluent component is present in an amount of from about 10% to about 50% by weight of the formulation. In some embodiments, the diluent component is present in an amount of from about 20% to about 60% by weight of the formulation. In some embodiments, the diluent component is present in an amount of from about 20% to about 50% by weight of the formulation. In some embodiments, the diluent component is present in an amount of from about 20% to about 40% by weight of the formulation. In some embodiments, the diluent component is present in an amount of about 35% by weight of the formulation.

In some embodiments, the diluent component comprises microcrystalline cellulose, mannitol, lactose, sucrose, dextrose, starch, sorbitol, dibasic calcium phosphate, cellulose, hydroxypropyl cellulose, xylitol, sodium carbonate, calcium phosphate, hydroxypropyl methylcellulose, hydroxypropyl methylcellulose acetate stearate, calcium sulfate, calcium lactate, calcium carbonate, sodium chloride, povidone, or a clay, or a mixture of any of the aforementioned.

In some embodiments, the diluent component comprises microcrystalline cellulose, mannitol, lactose, sucrose, starch, sorbitol, dibasic calcium phosphate, lactose monohydrate, spray-dried monohydrate lactose, lactose-316 Fast Flo®, acidified cellulose, starch 1500, prosolve MCC, colloidal silica, saccharides, disaccharides, polysaccharides, cellulose, cellulose ethers, hydroxypropyl cellulose, sugar alcohols, xylitol, sodium carbonate, dicalcium phosphate, compressible sugars, dibasic calcium phosphate dehydrate, tribasic calcium phosphate, dextrose, dicalcium phosphate dihydrate, calcium phosphate, anhydrous lactose, spray-dried lactose, pregelatinized starch, compressible sugar, hydroxy propylmethylcellulose, hydroxypropylmethylcellulose acetate stearate, sucrose-based diluents, confectioner's sugar, monobasic calcium sulfate monohydrate, calcium sulfate dihydrate; calcium lactate trihydrate, dextrates, hydrolyzed cereal solids, amylose, powdered cellulose, calcium carbonate, glycine, kaolin, sodium chloride; inositol, or bentonite, or a mixture of any of the aforementioned.

In some embodiments, the diluent component comprises microcrystalline cellulose and mannitol.

In some embodiments, the diluent component comprises at least 10% microcrystalline cellulose by weight. In some embodiments, the diluent component comprises at least 25% microcrystalline cellulose by weight. In some embodiments, the diluent component comprises at least 50% microcrystalline cellulose by weight. In some embodiments, the diluent component comprises from about 60% to about 65% microcrystalline cellulose by weight. In some embodiments, the diluent component comprises about 63% microcrystalline cellulose by weight.

In some embodiments, the diluent component comprises at least 10% mannitol by weight. In some embodiments, the diluent component comprises at least 20% mannitol by weight. In some embodiments, the diluent component comprises at least 30% mannitol by weight. In some embodiments, the diluent component comprises from about 30% to about 40% mannitol by weight.

In some embodiments, the diluent component comprises about 37% mannitol by weight.

In some embodiments, the diluent component comprises microcrystalline cellulose and mannitol wherein the ratio of microcrystalline cellulose to mannitol is about 1:1 to about 2:1.

In some embodiments, the diluent component comprises microcrystalline cellulose and mannitol wherein the ratio of microcrystalline cellulose to mannitol is about 1.2:1 to about 1.8:1.

In some embodiments, the diluent component comprises at least one diluent having a moisture content of not more than about 2% by weight of the diluent. In some embodiments, the diluent component comprises at least one diluent having a moisture content of not more than about 1.5% by weight of the diluent. In some embodiments, the at least one diluent comprises microcrystalline cellulose. In some embodiments, the at least one diluent comprises mannitol. In some embodiments, the at least one diluent comprises microcrystalline cellulose and mannitol.

In some embodiments, the pharmaceutical formulation further comprises a disintegrant component.

In some embodiments, the disintegrant component is present in an amount of from about 0.5% to about 15% by weight of the formulation. In some embodiments, the disintegrant component is present in an amount of from about 1% to about 15% by weight of the formulation. In some embodiments, the disintegrant component is present in an amount of from about 1% to about 10% by weight of the formulation. In some embodiments, the disintegrant component is present in an amount of from about 2% to about 10% by weight of the formulation. In some embodiments, the disintegrant component is present in an amount of from about 4% to about 8% by weight of the formulation. In some embodiments, the disintegrant component is present in an amount of about 6% by weight of the formulation. In some embodiments, the disintegrant component is present in an amount of about 8% by weight of the formulation.

In some embodiments, the disintegrant component comprises sodium starch glycolate, croscarmellose sodium, povidone, crospovidone, pregelatinized starch, starch, guar gum, an alginate, an ion-exchange resin, a clay, talc, methyl cellulose, ethyl cellulose, calcium carbonate, carboxymethylcellulose calcium, carboxymethyl cellulose sodium, or agar, or a mixture of any of the aforementioned.

In some embodiments, the disintegrant component comprises sodium starch glycolate, croscarmellose sodium, povidone, crospovidone, pregelatinized starch, starch, guar gum, an alginate, an ion-exchange resin, a clay, talc, methyl cellulose, ethyl cellulose, calcium carbonate, carboxymethylcellulose calcium, carboxymethyl cellulose sodium, agar, an ion-exchange resin, cellulose, hydroxypropyl cellulose, low substituted hydroxypropyl cellulose, corn starch, microcrystalline cellulose, modified corn starch, sodium carboxymethyl starch, pregelatinized starch, alginic acid, hydroxypropyl methylcellulose (HPMC), low substituted hydroxypropyl cellulose (L-HPC), lactose, magnesium aluminum silicate, methylcellulose, polacrilin potassium, or sodium alginate, or a mixture of any of the aforementioned.

In some embodiments, the disintegrant component comprises sodium starch glycolate. In some embodiments, the disintegrant component comprises sodium starch glycolate, croscarmellose sodium, crospovidone, or pregelatinized starch, or a mixture of any of the aforementioned.

In some embodiments, the pharmaceutical formulation further comprises a glidant component.

In some embodiments, the glidant component is present in an amount of from about 0.01% to about 5% by weight of the formulation. In some embodiments, the glidant component is present in an amount of from about 0.1% to about 2% by weight of the formulation. In some embodiments, the glidant component is present in an amount of from about 0.1% to about 1% by weight of the formulation. In some embodiments, the glidant component is present in an amount of about 0.5% by weight of the formulation.

In some embodiments, the glidant component comprises silicon dioxide, talc, starch, a silicate, a clay, or a mixture of any of the aforementioned.

In some embodiments, the glidant component comprises colloidal silicon dioxide, talc, starch, or corn starch, or a mixture of any of the aforementioned. In some embodiments, the glidant component comprises colloidal silicon dioxide.

In some embodiments, the pharmaceutical formulation further comprises a lubricant component.

In some embodiments, the lubricant component is present in an amount of from about 0.01% to about 5% by weight of the formulation. In some embodiments, the lubricant component is present in an amount of from about 0.5% to about 2% by weight of the formulation. In some embodiments, the lubricant component is present in an amount of from about 0.5% to about 1.5% by weight of the formulation. In some embodiments, the lubricant component is present in an amount of about 1% by weight of the formulation.

In some embodiments, the lubricant component comprises magnesium stearate, sodium stearyl fumarate, stearic acid, a hydrogenated oil, an alginate, polyethylene glycol, glyceryl monostearate, glyceryl palmitostearate, glyceryl behenate, calcium stearate, talc, starch, a metal lauryl sulfate, mineral oil, a fatty acid, a poloxamer, or a metal stearate, or a mixture of any of the aforementioned.

In some embodiments, the lubricant component comprises magnesium stearate, sodium stearyl fumarate, stearic acid, a hydrogenated oil, polyethylene glycol, glyceryl behenate, silica, fats, calcium stearate, talc, solubilizers such as fatty acids, glyceryl behenate, glycerin monostearate, glyceryl palmitostearate, magnesium lauryl sulfate, mineral oil, palmitic acid, myristic acid, poloxamer, sodium benzoate, sodium chloride, sodium lauryl sulfate, zinc stearate, or potassium benzoate, or a mixture of any of the aforementioned.

In some embodiments, the lubricant component comprises magnesium stearate, sodium stearyl fumarate, stearic acid, a hydrogenated oil, polyethylene glycol, or glyceryl behenate, or a mixture of any of the aforementioned. In some embodiments, the lubricant component comprises magnesium stearate.

In some embodiments, the pharmaceutical formulation further comprises a binder component.

In some embodiments, the binder component is present in an amount of from about 0.1% to about 20% by weight of the formulation. In some embodiments, the binder component is present in an amount of from about 0.1% to about 5% by weight of the formulation. In some embodiments, the binder component is present in an amount of from about 1% to about 5% by weight of the formulation. In some embodiments, the binder component is present in an amount of from about 1% to about 3% by weight of the formulation. In some embodiments, the binder component is present in an amount of about 2% by weight of the formulation.

In some embodiments, the binder component comprises hydroxypropyl cellulose, hydroxypropyl methylcellulose, povidone, copovidone, poly(vinyl pyrrolidone-co-vinyl acetate), tragacanth, acacia, starch, sodium starch, methyl cellulose, ethyl cellulose, gelatin, glucose, carboxymethylcellulose calcium, or carboxymethylcellulose sodium, or a mixture of any of the aforementioned.

In some embodiments, the binder component comprises hydroxypropyl cellulose, povidone, poly(vinyl pyrrolidone-co-vinyl acetate), tragacanth, acacia, starch, sodium starch, hydroxypropyl methylcellulose, copovidone, methyl cellulose, ethyl cellulose, microcrystalline cellulose, hydroxypropyl methylcellulose carboxyvinyl polymer, carboxymethylcellulose calcium, carboxymethylcellulose sodium, ceratonia, chitosan, cottonseed oil, dextrates, dextrin, gelatin, glucose, glyceryl behenate, galactomannan polysaccharide, hydroxyethyl cellulose, hydroxyethylmethyl cellulose, inulin, lactose, magnesium aluminum silicate, maltodextrin, methylcellulose, a poloxamer, polycarbophil, polydextrose, polyethylene glycol, polyethylene oxide, polymethacrylates, sodium alginate, sorbitol, starch, sucrose, sunflower oil, vegetable oil, tocofersolan, or zein, or a mixture of any of the aforementioned.

In some embodiments, the binder component comprises hydroxypropyl cellulose, povidone, or poly(vinyl pyrrolidone-co-vinyl acetate), or a mixture of any of the aforementioned. In some embodiments, the binder component comprises hydroxypropyl cellulose.

In some embodiments, the formulation stability of the pharmaceutical formulation at a temperature of about 25° C. and about 60% relative humidity is comparable to the formulation stability of a formulation comprising a compound of Formula (I), or a pharmaceutically acceptable salt thereof, without the stabilizing agent at a refrigeration temperature. In some embodiments, the six month stability of the pharmaceutical formulation at a temperature of about 25° C. and about 60% relative humidity is comparable to the six month stability of a formulation comprising a compound of Formula (I), or a pharmaceutically acceptable salt thereof, without the stabilizing agent at a refrigeration temperature. In some embodiments, the formulation stability of the pharmaceutical formulation at a temperature of about 25° C. and about 60% relative humidity is improved compared with the formulation stability of a formulation comprising a compound of Formula (I), or a pharmaceutically acceptable salt thereof, without the stabilizing agent under the same conditions. In some embodiments, the six month stability of the pharmaceutical formulation at a temperature of about 25° C. and about 60% relative humidity is improved compared with the six month stability of a formulation comprising a compound of Formula (I), or a pharmaceutically acceptable salt thereof, without the stabilizing agent under the same conditions. In some embodiments, the pharmaceutical formulation is at least twice as stable as a formulation comprising a compound of Formula (I), or a pharmaceutically acceptable salt thereof, without the stabilizing agent after storage at a temperature of about 25° C. and about 60% relative humidity for six months. In some embodiments, the pharmaceutical formulation is twice as stable as a formulation comprising a compound of Formula (I), or a pharmaceutically acceptable salt thereof, without the stabilizing agent after storage at a temperature of about 25° C. and about 60% relative humidity for six months. In some embodiments, the formulation stability of the pharmaceutical formulation at a refrigeration temperature is improved compared with the formulation stability of a formulation comprising a compound of Formula (I), or a pharmaceutically acceptable salt thereof, without the stabilizing agent under the same conditions. In some embodiments, the six month stability of the pharmaceutical formulation at a refrigeration temperature is improved compared with the six month stability of a formulation comprising a compound of Formula (I), or a pharmaceutically acceptable salt thereof, without the stabilizing agent under the same conditions. In some embodiments, the pharmaceutical formulation is at least twice as stable as a formulation comprising a compound of Formula (I), or a pharmaceutically acceptable salt thereof, without the stabilizing agent after storage at a refrigeration temperature for six months. In some embodiments, the pharmaceutical formulation is twice as stable as a formulation comprising a compound of Formula (I), or a pharmaceutically acceptable salt thereof, without the stabilizing agent after storage at a refrigeration temperature for six months. In some embodiments, the stability is measured after a period of six months. As used herein, “six month stability” means after storage for six months under the specified conditions of temperature and humidity.

Compound 1, or a pharmaceutically acceptable salt thereof, can degrade to form impurities. Six major degradants that may be formed are Compounds 2-7, which are listed in Tables A and 4-7. As used herein, “major degradants” refers to Compounds 2-7.

In some embodiments, the total amount (%) of major degradants of the pharmaceutical formulation after storage at a temperature of about 25° C. and about 60% relative humidity for six months is comparable to the total amount (%) of major degradants of a formulation comprising a compound of Formula (I), or a pharmaceutically acceptable salt thereof, without the stabilizing agent after storage at a refrigeration temperature for six months. In some embodiments, the total amount (%) of major degradants of the pharmaceutical formulation is lower than the total amount (%) of major degradants of a formulation comprising a compound of Formula (I), or a pharmaceutically acceptable salt thereof, without the stabilizing agent after storage at a temperature of about 25° C. and about 60% relative humidity for six months. In some embodiments, the total amount of major degradants of the pharmaceutical formulation is about 50% or lower than the total amount of major degradants of a formulation comprising a compound of Formula (I), or a pharmaceutically acceptable salt thereof, without the stabilizing agent after storage at a temperature of about 25° C. and about 60% relative humidity for six months. In some embodiments, the total amount (%) of major degradants of the pharmaceutical formulation is lower than the total amount (%) of major degradants of a formulation comprising a compound of Formula (I), or a pharmaceutically acceptable salt thereof, without the stabilizing agent after storage at a refrigeration temperature for six months. In some embodiments, the total amount of major degradants of the pharmaceutical formulation is about 50% or lower than the total amount of major degradants of a formulation comprising a compound of Formula (I), or a pharmaceutically acceptable salt thereof, without the stabilizing agent after storage at a refrigeration temperature for six months.

In some embodiments, the refrigeration temperature is from about 2° C. to about 8° C.

In some embodiments, the pharmaceutical formulation does not require cold chain storage.

The present application also relates to a solid dosage form comprising a pharmaceutical formulation provided herein.

In some embodiments, the solid dosage form is suitable for oral administration.

In some embodiments, the dosage form provided herein is in the form of tablets, capsules, pills, powders, sachets, and soft and hard gelatin capsules. In other embodiments, the dosage form provided herein is in the form of a capsule.

In some embodiments, the dosage form provided herein is in the form of a tablet or capsule. In some embodiments, the dosage form provided herein is in the form of a tablet.

In preparing a formulation, Compound 1 can be milled to provide the appropriate particle size prior to combining with the other ingredients. Compound 1 can be milled to a particle size of less than 200 mesh. The particle size can be adjusted by milling to provide a substantially uniform distribution in the formulation, e.g., about 40 mesh.

Compound 1 may be milled using known milling procedures such as wet milling to obtain a particle size appropriate for tablet formation and for other formulation types. Finely divided (nanoparticulate) preparations of the compounds disclosed herein can be prepared by processes known in the art, e.g., see International App. No. WO 2002/000196.

The present disclosure further provides a dosage form which comprises any of the above-described formulations of the disclosure. In some embodiments, the dosage form is a solid dosage form, such as a tablet or capsule.

For preparing solid dosage forms such as tablets, Compound 1, or a pharmaceutically acceptable salt thereof, can be mixed with excipients to form a solid preformulation formulation containing a homogeneous mixture of Compound 1, or a pharmaceutically acceptable salt thereof. When referring to these preformulation formulations as homogeneous, the active ingredient is typically dispersed evenly throughout the formulation so that the formulation can be readily subdivided into equally effective unit dosage forms such as tablets, pills and capsules. This solid preformulation is then subdivided into unit dosage forms of the type described above containing from, for example, about 0.1 to about 1000 mg of Compound 1.

In some embodiments, film-coating agents can be present in an amount of 0% to about 5% by weight. In some embodiments, film-coating agents can be present in an amount of 0.1% to about 5% by weight. In some embodiments, film-coating agents can be present in an amount of 1% to about 5% by weight. In some embodiments, a film-coating agent is present in an amount of about 4% by weight. Non-limiting illustrative examples of film-coating agents include hypromellose or polyvinyl alcohol based coating with titanium dioxide, talc and optionally colorants available in several commercially available complete coating systems. In some embodiments, the film-coating agent is Opadry AMB II.

In some embodiments, the administration is oral.

Provided herein are oral dosage forms comprising a pharmaceutical formulation of the disclosure.

In some embodiments, the compound of Formula (I) or a pharmaceutically acceptable salt thereof is present in the oral dosage form in an amount of about 400 mg to about 800 mg. In some embodiments, the compound of Formula (I) or a pharmaceutically acceptable salt thereof is present in the oral dosage form in an amount of about 200 mg to about 600 mg. In some embodiments, the compound of Formula (I) or a pharmaceutically acceptable salt thereof is present in the oral dosage form in an amount of about 300 mg to about 500 mg. In some embodiments, the compound of Formula (I) or a pharmaceutically acceptable salt thereof is present in the oral dosage form in an amount of about 350 mg to about 450 mg. In some embodiments, the compound of Formula (I) or a pharmaceutically acceptable salt thereof is present in the oral dosage form in an amount of about 400 mg. In some embodiments, the compound of Formula (I) or a pharmaceutically acceptable salt thereof is present in the oral dosage form in an amount of about 500 mg. In some embodiments, the compound of Formula (I) or a pharmaceutically acceptable salt thereof is present in the oral dosage form in an amount of about 600 mg. In some embodiments, the compound of Formula (I) or a pharmaceutically acceptable salt thereof is present in the oral dosage form in an amount of about 700 mg. In some embodiments, the compound of Formula (I) or a pharmaceutically acceptable salt thereof is present in the oral dosage form in an amount of about 800 mg.

In some embodiments, the oral dosage form is a tablet.

In some embodiments, the oral dosage form is a capsule.

In some embodiments, the oral dosage form further comprises an outer coating.

In some embodiments, the outer coating is a film-coating agent.

The pharmaceutical formulations in solid dosage form provided herein which are suitable for oral administration can be prepared by blending Compound 1 ((R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid) with an organic acid. The pharmaceutical formulation formed can be further compressed to form a tablet. In some embodiments, the organic acid is fumaric acid.

The pharmaceutical formulations in solid dosage form provided herein which are suitable for oral administration can be prepared by blending Compound 1 ((R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid) with an organic acid and one or more portions of a diluent. The pharmaceutical formulation formed can be further compressed to form a tablet. In some embodiments, the organic acid is fumaric acid and the diluent is microcrystalline cellulose and mannitol.

The pharmaceutical formulations in solid dosage form provided herein which are suitable for oral administration can be prepared by:

a) blending Compound 1 ((R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid) with an organic acid to form a first mixture;

b) blending the first mixture with a diluent to form a second mixture;

c) blending the second mixture with a binder, a disintegrant, and a glidant to form a third mixture; and

d) blending the third mixture with a lubricant to form the pharmaceutical formulation. The pharmaceutical formulation formed can be further compressed to form a tablet. In some embodiments, the organic acid is fumaric acid, the diluent is microcrystalline cellulose and mannitol, the binder is hydroxypropyl cellulose, the disintegrant is hydroxypropyl cellulose and sodium starch glycolate, the glidant is colloidal silicon dioxide, and the lubricant is magnesium stearate.

Compound 1 ((R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid), diluent such as microcrystalline cellulose and mannitol, organic acid such as fumaric acid, or mixtures thereof can be prescreened to a uniformed particle size, for example, between 40 and 100 mesh prior to subject the each of the blending steps in the process of making the pharmaceutical formulations or tablets. In some embodiments, the particle size is 30, 40, 60, 70 or 80 mesh.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the disclosure (e.g., Compound 1 and organic acids) may be milled using known milling procedures. Finely divided (nanoparticulate) preparations of the compounds of the disclosure can be prepared by processes known in the art see, e.g., WO 2002/000196.

In some embodiments, a dry granulation process is used to produce the formulation.

The formulations can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient.

In some embodiments, the excipients used to formulate the pharmaceutical formulations are of high purity and are substantially free of potentially harmful contaminants (e.g., at least National Food grade, generally at least analytical grade, and more typically at least pharmaceutical grade). Particularly for human consumption, the formulation is preferably manufactured or formulated under Good Manufacturing Practice standards as defined in the applicable regulations of the U.S. Food and Drug Administration.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the formulations comprising a compound of Formula (I), or the pharmaceutically acceptable salt thereof, can be combined in any suitable combination.

At various places in the present specification, certain features of the formulations of Compound 1 are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

In some embodiments, the components are present in exactly the ranges or amounts specified (e.g., the term “about” is not present).

As used herein, the term “about” for amounts or percentages of API or excipients means plus or minus 10% of the value.

In some embodiments, the term “about” in the context of temperature means±3° C.

At various places in the present specification, variables defining divalent linking groups may be described. It is specifically intended that each linking substituent include both the forward and backward forms of the linking substituent. For example, —NR(CR′R″)ₙ— includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” then it is understood that the “alkyl” represents a linking alkylene group.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkoxy”, employed alone or in combination with other terms, refers to a group of formula —O-alkyl, wherein the alkyl group is as defined above. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms.

The term “alkyl” employed alone or in combination with other terms, refers to a saturated hydrocarbon group that may be straight-chained or branched. The term “Cₙ₋ₘ alkyl”, refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

As used herein, the term “alkylated phenol” means a compound which is a phenol, which is optionally fused to a 5-6 membered cycloalkyl or heterocyclyl ring, wherein the optionally fused phenol is substituted by 1, 2, 3, 4, 5, or 6 independently selected substituents selected from C₁₋₁₈ alkyl and C₁₋₆ alkoxy. Examples of an alkylated phenol include butylated hydroxyanisole, butylated hydroxytoluene, tocopherol and the like.

The term “alkylene”, employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl and the like.

As used herein, the term “antioxidant” means a substance that inhibits oxidation or inhibits reactions promoted by, for example, oxygen, peroxides or free radicals.

As used herein, the term “carboxylic acid group”, employed alone or in combination with other terms, refers to a C(═O)OH group, which also may be written as C(O)OH, COOH or CO₂H.

As used herein, the term “Cₙ₋ₘ alkyl carboxylic acid” refers to an alkyl group having n to m carbon atoms and substituted with at least one carboxylic acid group. In some embodiments, the Cₙ₋ₘ alkyl carboxylic acid is optionally substituted by 1, 2, 3, 4, 5, or 6 substituents independently selected from SH, NH₂, OH, and CO₂H. Example Cₙ₋ₘ alkyl carboxylic acids include citric acid, succinic acid, adipic acid, malonic acid, glutaric acid, gluconic acid, lactic acid, glycolic acid, malic acid, tartaric acid, tartronic acid, galactaric acid, glutamic acid, aspartic acid and the like. In some embodiments, the Cₙ₋ₘ alkyl carboxylic acid has 1 to 8, 1 to 6, 1 to 4, or 1 to 3 carbon atoms. In some embodiments, the Cₙ₋ₘ alkyl carboxylic acid is substituted with 1 or 2 carboxylic acid groups.

As used herein, the term “Cₙ₋ₘ alkenyl carboxylic acid” refers to an alkenyl group having n to m carbon atoms and substituted with at least one carboxylic acid group. In some embodiments, the Cₙ₋ₘ alkenyl carboxylic acid is optionally substituted by 1, 2, 3, 4, 5, or 6 substituents independently selected from SH, NH₂, OH, and CO₂H. Example Cₙ₋ₘ alkenyl carboxylic acids include fumaric acid, maleic acid, sorbic acid and the like. In some embodiments, the Cₙ₋ₘ alkenyl carboxylic acid has 1 to 8, 1 to 6, 1 to 4, or 1 to 3 carbon atoms. In some embodiments, the Cₙ₋ₘ alkenyl carboxylic acid is substituted with 1 or 2 carboxylic acid groups.

As used herein, the term “phenyl carboxylic acid” refers to a phenyl group substituted with at least one carboxylic acid group. In some embodiments, the phenyl carboxylic acid is optionally substituted by 1, 2, 3, 4 or 5 substituents independently selected from SH, NH₂, OH, and CO₂H. Example phenyl carboxylic acids include benzoic acid, phthalic acid, isophthalic acid, terephthalic acid, trimelitic acid and the like. In some embodiments, the phenyl carboxylic acid is substituted with 1 or 2 carboxylic acid groups.

As used herein, the term “n-m membered,” where n and m are each an integer, typically describes the number of ring-forming atoms in a moiety where the number of ring-forming atoms ranges from n to m. For example, piperidinyl, pyrazolyl and pyridyl are examples of a 5-6 membered heteroaryl ring.

As used herein, the term “5-6 membered heteroaryl carboxylic acid” refers to a 5-6 membered heteroaryl group substituted with at least one carboxylic acid group. In some embodiments, the 5-6 membered heteroaryl carboxylic acid is optionally substituted by 1, 2, 3, 4, 5, or 6 substituents independently selected from SH, NH₂, OH, and CO₂H. In some embodiments, the 5-6 membered heteroaryl carboxylic acid is substituted with 1 or 2 carboxylic acid groups.

As used herein, the term “4-7 membered heterocyclyl carboxylic acid” refers to a 4-7 membered heterocyclyl group substituted with at least one carboxylic acid group. In some embodiments, the 4-7 membered heterocyclyl carboxylic acid is optionally substituted by 1, 2, 3, 4, 5, or 6 substituents independently selected from SH, NH₂, OH, and CO₂H. In some embodiments, the 4-7 membered heterocyclyl carboxylic acid is substituted with 1 or 2 carboxylic acid groups.

As used herein, the term “C₃₋₇ cyclohexyl carboxylic acid” refers to a C₃₋₇ cyclohexyl group substituted with at least one carboxylic acid group. In some embodiments, the C₃₋₇ cyclohexyl carboxylic acid is optionally substituted by 1, 2, 3, 4, 5, or 6 substituents independently selected from SH, NH₂, OH, and CO₂H. In some embodiments, the C₃₋₇ cyclohexyl carboxylic acid is substituted with 1 or 2 carboxylic acid groups.

As used herein, the term “cold chain storage” refers to the maintenance of refrigerated temperatures for formulations and dosage forms from the time they are manufactured through their shipment and delivery to health care facilities until their administration to patients. In some embodiments, the formulations and dosage forms in cold chain storage are maintained at a temperature of less than 0° C.

As used herein, the term “compound,” as used herein is meant to include all stereoisomers, geometric isomers, tautomers and isotopes of the structures depicted. The term is also meant to refer to compounds described herein (e.g., Compound 1, stabilizing agents, organic acids), regardless of how they are prepared, e.g., synthetically, through biological process (e.g., metabolism or enzyme conversion), or a combination thereof.

As used herein, the term “Cₙ₋ₘ” indicates a range which includes the endpoints, wherein n and m are integers and indicate the number of carbons. Examples include C₁₋₄, C₁₋₆ and the like.

As used herein, the term “contacting” refers to the bringing together of indicated moieties in an in vitro system or an in vivo system. For example, “contacting” a PD1 or PD-L1 enzyme with a formulation disclosed herein includes the administration of a formulation disclosed herein to an individual or patient, such as a human, having a PD1 or PD-L1 enzyme, as well as, for example, introducing a formulation disclosed herein into a sample containing a cellular or purified preparation containing the PD1 or PD-L1 enzyme.

As used herein, the term “cyano” or “nitrile” refers to a group of formula —C≡N, which also may be written as —CN.

As used herein, the term “dosage form” refers to a physically discrete unit suitable as unitary dosages for human subjects and other mammals, each unit containing a predetermined quantity of active material calculated to produce the desired therapeutic effect, in association with a suitable pharmaceutical excipient.

As used herein, the term “ester” refers to an organic compound made by replacing the hydrogen of an acid by an alkyl or other organic group. Examples of esters of organic acids as described herein include acsorbyl palmitate, propyl gallate and the like.

As used herein, the term “granulating” refers the process where the powder particles are made into larger granules. Wet granulation refers to when granules are formed by the addition of a granulation liquid such as water to the mixture.

As used herein, the term “heteroatom” used herein is meant to include boron, phosphorus, sulfur, oxygen and nitrogen.

As used herein, the term “heteroaryl”, employed alone or in combination with other terms, refers to a monocyclic aromatic heterocycle having at least one heteroatom ring member selected from boron, phosphorus, sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring.

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl and pyridazinyl.

As used herein, the term “cycloalkyl,” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6, or 7 ring-forming carbons (C₃₋₇). In some embodiments, the cycloalkyl group has 3 to 7 members, 3 to 6 ring members, 3 to 5 ring members, or 3 to 4 ring members. In some embodiments, the cycloalkyl group is monocyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, and the like. In some embodiments, the cycloalkyl group is cyclopropyl, cyclobutyl, cyclopentyl, or cyclohexyl.

As used herein, the term “heterocycloalkyl,” employed alone or in combination with other terms, refers to a non-aromatic ring or ring system, which may optionally contain one or more alkenylene groups as part of the ring structure, which has at least one heteroatom ring member independently selected from boron, nitrogen, sulfur oxygen and phosphorus, and which has 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic or polycyclic (e.g., having two or three fused or bridged rings) ring systems or spirocycles. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Examples of heterocycloalkyl groups include azetidinyl, azepanyl, dihydrobenzofuranyl, dihydrofuranyl, dihydropyranyl, morpholino, piperidinyl, piperazinyl, oxopiperazinyl, pyranyl, pyrrolidinyl, tetrahydrofuranyl, tetrahydropyranyl, and the like.

As used herein, the terms “individual” or “patient,” used interchangeably, refer to any animal, including mammals, preferably mice, rats, other rodents, rabbits, dogs, cats, swine, cattle, sheep, horses, or primates, and most preferably humans.

As used herein, the term “inorganic acid salt” means a salt of an acid that does not contain C—H bonds. In some embodiments, the inorganic acid salt comprises an S(O)O⁻ group. Examples of an inorganic salt that comprises an S(O)O⁻ group include sodium sulfite, sodium bisulfite, sodium metabisulfite, sodium thiosulfate and the like.

As used herein, the term “organic acid” means an organic carboxylic acid selected from a C₁₋₁₈ alkyl carboxylic acid, a C₂₋₁₈ alkenyl carboxylic acid, a phenyl carboxylic acid, a 5-6 membered heteroaryl carboxylic acid, a 4-7 membered heterocyclyl carboxylic acid, or a C₃₋₇ cyclohexyl carboxylic acid. Examples of an organic acid include fumaric acid, citric acid, succinic acid, adipic acid, maleic acid, sorbic acid, malonic acid, glutaric acid, gluconic acid, lactic acid, glycolic acid, malic acid, tartaric acid, tartronic acid, galactaric acid, glutamic acid, aspartic acid, benzoic acid, phthalic acid, isophthalic acid, terephthalic acid, trimelitic acid, cysteine and the like.

As used herein, “an ester” of an “organic acid” is an organic acid as defined herein wherein the H atom of one carboxylic acid group is replaced with a group selected from C₁₋₈ alkyl, C₂₋₈ alkenyl, phenyl, 5-6 membered heteroaryl, 4-7 membered heterocyclyl, or C₃₋₇ cyclohexyl, wherein the C₁₋₈ alkyl, C₂₋₈ alkenyl, phenyl, 5-6 membered heteroaryl, 4-7 membered heterocyclyl, or C₃₋₇ cyclohexyl are each optionally substituted with 1, 2, 3, or 4 substituents independently selected from NH₂ and OH. Examples of esters of organic acids include, but are not limited to propyl gallate and ascorbyl palmitate.

As used herein, the term “metal sulfite” refers to a salt of formula S(O)O₂M₂, wherein M is an alkali metal counterion (e.g., Na+ or K+). An example of a metal sulfite is sodium sulfite.

As used herein, the term, “metal bisulfite” refers to a salt of formula HO—S(O)OM, wherein M is an alkali metal counterion (e.g., Na+ or K+). An example of a metal bisulfite is sodium bisulfite.

As used herein, the term “metal metabisulfite” refers to a salt of formula M₂S₂O₅, wherein M is an alkali metal counterion (e.g., Na+ or K+). An example of a metal metabisulfite is sodium metabisulfite.

As used herein, the term “metal thiosulfate” refers to a salt of formula M₂S₂O₃, wherein M is an alkali metal counterion (e.g., Na+ or K+). An example of a metal thiosulfate is sodium thiosulfate.

As used herein, the term “metal formaldehyde sulfoxylate” refers to a salt of formula HO—CH₂—S(O)OM, wherein M is an alkali metal counterion (e.g., Na+ or K+). An example of a metal formaldehyde sulfoxylate is sodium formaldehyde sulfoxylate.

As used herein, the term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

As used herein, the phrase “pharmaceutically acceptable” is employed herein to refer to those compounds, materials, formulations and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The present invention also includes formulations of pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compounds disclosed herein include the non-toxic salts of the parent compound formed, e.g., from non-toxic inorganic or organic acids. The pharmaceutically acceptable salts of the compounds disclosed herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002). In some embodiments, the compounds described herein include the N-oxide forms.

As used herein, the term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

As used herein, the term “sulfido” refers to a sulfur atom as a divalent substituent, forming a thiocarbonyl group (C═S) when attached to carbon.

Compound 1 or a pharmaceutically acceptable salt thereof can also be in a solvated form. The term “solvate” means a solid form that includes solvent molecules with Compound 1 or a pharmaceutically acceptable salt thereof. A solvate where the solvent is water is generally referred to as a “hydrate” or “hydrated form.”

As used herein, the term “stabilizing agent” refers to a substance or mixture of substances that improves the stability of the pharmaceutical formulation and/or the compatibility of the components in the formulation. In some embodiments, the stabilizing agent is an antioxidant.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds or salts are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

As used herein, the term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

All compounds, and pharmaceutically acceptable salts thereof, described herein can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

### II. Synthesis

Compound 1, including salts thereof, can be prepared using known organic synthesis techniques and can be synthesized according to any of numerous possible synthetic routes. Compound 1 was synthesized and tested as described in U.S. Pat. No. 10,308,644 and US 2018/0177870, each of which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC) or thin layer chromatography (TLC).

### III. Uses of the Compounds

Formulations of the present disclosure can inhibit the activity of PD-1/PD-L1 protein/protein interaction and, thus, are useful in treating diseases and disorders associated with activity of PD-1 and the diseases and disorders associated with PD-L1 including its interaction with other proteins such as PD-1 and B7-1 (CD80). In certain embodiments, the formulations of the present disclosure are useful for therapeutic administration to enhance, stimulate and/or increase immunity in cancer, chronic infection or sepsis, including enhancement of response to vaccination. In some embodiments, the present disclosure provides a method for inhibiting the PD-1/PD-L1 protein/protein interaction. The method includes administering to an individual or a patient in need thereof a pharmaceutical formulation comprising a compound of Formula (I), or a pharmaceutically acceptable salt thereof. The formulations of the present disclosure can be used alone, in combination with other agents or therapies or as an adjuvant or neoadjuvant for the treatment of diseases or disorders, including cancer or infection diseases. For the uses described herein, any of the formulations of the disclosure, including any of the embodiments thereof, may be used. In some embodiments is a method of inhibiting the activity of PD-1/PD-L1 protein/protein interaction, comprising administering to a patient in need thereof an oral dosage form disclosed herein.

The formulations of the present disclosure inhibit the PD-1/PD-L1 protein/protein interaction, resulting in a PD-1 pathway blockade. The blockade of PD-1 can enhance the immune response to cancerous cells and infectious diseases in mammals, including humans. In some embodiments, the present disclosure provides treatment of an individual or a patient in need thereof in vivo using formulation comprising a compound of Formula (I) or a pharmaceutically acceptable salt thereof such that growth of cancerous tumors is inhibited. A pharmaceutical formulation comprising a compound of Formula (I), or a pharmaceutically acceptable salt thereof, can be used to inhibit the growth of cancerous tumors. Alternatively, pharmaceutical formulation comprising a compound of Formula (I), or a pharmaceutically acceptable salt thereof, can be used in conjunction with other agents or standard cancer treatments, as described below. In one embodiment, the present disclosure provides a method for inhibiting growth of tumor cells in vitro. The method includes contacting the tumor cells in vitro with a pharmaceutical formulation comprising a compound of Formula (I), or of a pharmaceutically acceptable salt thereof. In another embodiment, the present disclosure provides a method for inhibiting growth of tumor cells in an individual or a patient in need thereof. The method includes administering to the individual or patient in need thereof a therapeutically effective amount of a pharmaceutical formulation comprising a compound of Formula (I), or a pharmaceutically acceptable salt. In some embodiments is a method of inhibiting PD-1/PD-L1 protein/protein interaction, comprising administering to a patient in need thereof an oral dosage form disclosed herein. In some embodiments is a method of decreasing or reducing the interaction of PD-1 and PD-L1, comprising administering to a patient in need thereof an oral dosage form disclosed herein.

In some embodiments, provided herein is a method for treating cancer. The method includes administering to a patient in need thereof, a therapeutically effective amount of a pharmaceutical formulation comprising a compound of Formula (I), or a pharmaceutically acceptable salt thereof. Examples of cancers include those whose growth may be inhibited using compounds of the disclosure and cancers typically responsive to immunotherapy.

In some embodiments, the present disclosure provides a method of enhancing, stimulating and/or increasing the immune response in a patient in need thereof. The method includes administering to the patient in need thereof a therapeutically effective amount of a pharmaceutical formulation comprising a compound of Formula (I), or a pharmaceutically acceptable salt thereof. In some embodiments is a method of enhancing, stimulating and/or increasing an immune response in a patient in need thereof, comprising administering to the patient in need thereof an oral dosage form disclosed herein. In some embodiments, the immune response is a T cell immune response. In some embodiments, the T cell immune response is a cytotoxic or effector T cell response.

In some embodiments is a method of treating a PD-1-related disease or condition, comprising administering to a patient in need thereof an oral dosage form disclosed herein. In some embodiments, the disease or condition is an infection disease, inflammation, autoimmune disease, cancer, or neurodegenerative disorder.

Examples of cancers that are treatable using the formulations of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers, especially metastatic cancers that express PD-Ll.

In some embodiments, cancers treatable with formulations of the present disclosure include melanoma (e.g., metastatic malignant melanoma, cutaneous melanoma), renal cancer (e.g., clear cell carcinoma), prostate cancer (e.g., hormone refractory prostate adenocarcinoma), breast cancer (e.g., breast invasive carcinoma), colon cancer, lung cancer (e.g., non-small cell lung cancer and small cell lung cancer), squamous cell head and neck cancer (e.g., squamous cell carcinoma of the head and neck), urothelial cancer (e.g., bladder cancer, nonmuscle invasive bladder cancer (NMIBC)) and cancers with high microsatellite instability (MSI^{high}). Additionally, the disclosure includes refractory or recurrent malignancies whose growth may be inhibited using the compounds of the disclosure.

In some embodiments, cancers that are treatable using the formulations of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In some embodiments, cancers that are treatable using the formulations of the present disclosure include, but are not limited to, cholangiocarcinoma, bile duct cancer, biliary tract cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the formulations of the present disclosure can be used to treat sickle cell disease and sickle cell anemia.

In some embodiments, diseases and indications that are treatable using the formulations of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, hamartoma, and teratoma.

Exemplary lung cancers include non-small cell lung cancer (NSCLC) (e.g., squamous cell NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, and mesothelioma.

Exemplary gastrointestinal cancers include cancers of the esophagus (carcinoma, squamous cell carcinoma, adenocarcinoma, leiomyosarcoma, lymphoma), stomach (carcinoma, lymphoma, leiomyosarcoma, adenocarcinoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer (e.g., colorectal adenocarcinoma).

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma). In some embodiments, the cancer is a urological cancer (e.g., papillary kidney carcinoma, testicular germ cell cancer, chromophobe renal cell carcinoma, clear cell renal carcinoma, or prostate adenocarcinoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors.

Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, serous adenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma (e.g., cutaneous squamous cell carcinoma), Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

PD-1 pathway blockade with formulations of the present disclosure can also be used for treating infections such as viral, bacteria, fungus and parasite infections. The present disclosure provides a method for treating infections such as viral infections. The method includes administering to a patient in need thereof, a therapeutically effective amount of a pharmaceutical formulation comprising a compound of Formula (I), or a pharmaceutically acceptable salt thereof. Examples of viruses causing infections treatable by methods of the present disclosure include, but are not limit to, human immunodeficiency virus, human papillomavirus, influenza, hepatitis A, B, C or D viruses, adenovirus, poxvirus, herpes simplex viruses, human cytomegalovirus, severe acute respiratory syndrome virus, ebola virus, and measles virus. In some embodiments, viruses causing infections treatable by methods of the present disclosure include, but are not limit to, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumpsvirus, rotavirus, measles virus, rubella virus, parvovirus, vaccinia virus, HTLV virus, dengue virus, papillomavirus, molluscum virus, poliovirus, rabies virus, JC virus, tuberculosis and arboviral encephalitis virus.

The present disclosure provides a method for treating bacterial infections. The method includes administering to a patient in need thereof, a therapeutically effective amount of a pharmaceutical formulation comprising a compound of Formula (I), or a pharmaceutically acceptable salt thereof. Non-limiting examples of pathogenic bacteria causing infections treatable by methods of the disclosure include chlamydia, rickettsial bacteria, mycobacteria, staphylococci, streptococci, pneumococci, meningococci and conococci, Klebsiella, Proteus, Serratia, Pseudomonas, Legionella, diphtheria, salmonella, bacilli, cholera, tetanus, botulism, anthrax, plague, leptospirosis, and Lyme's disease bacteria.

The present disclosure provides a method for treating fungus infections. The method includes administering to a patient in need thereof, a therapeutically effective amount of a pharmaceutical formulation comprising a compound of Formula (I), or a pharmaceutically acceptable salt thereof. Non-limiting examples of pathogenic fungi causing infections treatable by methods of the disclosure include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

The present disclosure provides a method for treating parasite infections. The method includes administering to a patient in need thereof, a therapeutically effective amount of a pharmaceutical formulation comprising a compound of Formula (I), or a pharmaceutically acceptable salt thereof. Non-limiting examples of pathogenic parasites causing infections treatable by methods of the disclosure include Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

The present disclosure provides a method for treating neurodegenerative diseases or disorders. The method includes administering to a patient in need thereof, a therapeutically effective amount of a pharmaceutical formulation comprising a compound of Formula (I), or a pharmaceutically acceptable salt thereof. Non-limiting examples of neurodegenerative diseases or disorders include Alzheimer's disease, Parkinson's disease, Huntington's disease, prion disease, Motor neurone diseases, Spinocerebellar ataxia and Spinal muscular atrophy.

It is believed that formulations comprising a compound of Formula (I), or a pharmaceutically acceptable salt thereof, or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

In some embodiments, the formulations disclosed herein are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

Cancer cell growth and survival can be impacted by dysfunction in multiple biological pathways. Thus, it may be useful to combine inhibitors of different mechanisms, such as enzyme inhibitors, signal transduction inhibitors, inhibitors of chromatin dynamics or modulators of immune responses, to treat such conditions. Targeting more than one signaling pathway (or more than one biological molecule involved in a given signaling pathway) may reduce the likelihood of drug-resistance arising in a cell population, or reduce the toxicity of treatment.

The formulations of the present disclosure can be used in combination with one or more other therapies for the treatment of diseases, such as cancer or infections. Examples of diseases and indications treatable with combination therapies include those as described herein. Examples of cancers include solid tumors and non-solid tumors, such as liquid tumors, blood cancers. Examples of infections include viral infections, bacterial infections, fungus infections or parasite infections. For example, the formulations of the present disclosure can be combined with one or more inhibitors of the following kinases for the treatment of cancer: Akt1, Akt2, Akt3, BCL2, CDK, TGF-βR, PKA, PKG, PKC, CaM-kinase, phosphorylase kinase, MEKK, ERK, MAPK, mTOR, EGFR, HER2, HER3, HER4, INS-R, IDH2, IGF-1R, IR-R, PDGFαR, PDGFβR, PI3K (alpha, beta, gamma, delta, and multiple or selective), CSF1R, KIT, FLK-II, KDR/FLK-1, FLK-4, fit-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the formulations of the present disclosure can be combined with one or more of the following inhibitors for the treatment of cancer or infections. Non-limiting examples of inhibitors that can be combined with the formulations of the present disclosure for treatment of cancer and infections include an FGFR inhibitor (FGFR1, FGFR2, FGFR3 or FGFR4, e.g., pemigatinib (INCY54828), INCB62079), a JAK inhibitor (JAK1 and/or JAK2, e.g., ruxolitinib, baricitinib or itacitinib (INCB39110)), an IDO inhibitor (e.g., epacadostat, NLG919, or BMS-986205, MK7162), an LSD1 inhibitor (e.g., INCB59872 and INCB60003), a TDO inhibitor, a PI3K-delta inhibitor (e.g., Parsaclisib (INCB50465) and INCB50797), a PI3K-gamma inhibitor such as PI3K-gamma selective inhibitor, a Pim inhibitor (e.g., INCB53914), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g., bevacizumab, pazopanib, sunitinib, sorafenib, axitinib, regorafenib, ponatinib, cabozantinib, axitinib, vandetanib, ramucirumab, lenvatinib, ziv-aflibercept), a PARP inhibitor (e.g., olaparib, rucaparib, veliparib, talazoparib, or niraparib), a CSF1R inhibitor, a TAM receptor tyrosine kinases (Tyro-3, Axl, and Mer), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), an arginase inhibitor (INCB001158), a PARP inhibitor (such as rucaparib or olaparib), sitravatinib, a B-Raf inhibitor-MEK inhibitor combination (such as encorafenib plus binimetinib, dabrafenib plus trametinib, or cobimetinib plus vemurafenib), and an adenosine receptor antagonist or combinations thereof.

In some embodiments, the formulations of the present disclosure can be combined with a TLR7 agonist (e.g., imiquimod).

The formulations of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, STING agonists, RIG-I agonists, oncolytic virotherapy and immunomodulating small molecules, including thalidomide or JAK1/2 inhibitor, PI3Kδ inhibitor and the like. The formulations can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat and zoledronate.

Other anti-cancer agent(s) include antibody therapeutics such as trastuzumab (Herceptin), antibodies to costimulatory molecules such as CTLA-4 (e.g., ipilimumab), 4-1BB (e.g., urelumab, utomilumab), antibodies to PD-1 and PD-L1, or antibodies to cytokines (IL-10, TGF-β, etc.). Examples of antibodies to PD-1 and/or PD-L1 that can be combined with compounds of the present disclosure for the treatment of cancer or infections such as viral, bacteria, fungus and parasite infections include, but are not limited to nivolumab, pembrolizumab, atezolizumab, durvalumab, avelumab and SHR-1210.

Formulations of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab or pembrolizumab. In some embodiments, the anti-PD1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab.

In some embodiments, the inhibitor of an immune checkpoint molecule is an inhibitor of LAG3, e.g., an anti-LAG3 antibody. In some embodiments, the anti-LAG3 antibody is BMS-986016, LAG525 or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an inhibitor of OX40, e.g., an anti-OX40 antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

The formulations of the present disclosure can further be used in combination with one or more anti-inflammatory agents, steroids, immunosuppressants or therapeutic antibodies.

The pharmaceutical formulations comprising a compound of Formula (I), or pharmaceutically acceptable salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The pharmaceutical formulations comprising a compound of Formula (I), or pharmaceutically acceptable salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the formulations of the present disclosure can be used in combination with tumor specific antigen such as heat shock proteins isolated from tumor tissue itself. In some embodiments, the pharmaceutical formulations comprising a compound of Formula (I), or pharmaceutically acceptable salts thereof can be combined with dendritic cells immunization to activate potent anti-tumor responses.

The formulations of the present disclosure can be used in combination with bispecific macrocyclic peptides that target Fe alpha or Fe gamma receptor-expressing effectors cells to tumor cells. The formulations of the present disclosure can also be combined with macrocyclic peptides that activate host immune responsiveness.

The formulations of the present disclosure can be used in combination with bone marrow transplant for the treatment of a variety of tumors of hematopoietic origin.

The pharmaceutical formulations comprising a compound of Formula (I), or pharmaceutically acceptable salts thereof can be used in combination with vaccines, to stimulate the immune response to pathogens, toxins, and self antigens. Examples of pathogens for which this therapeutic approach may be particularly useful, include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas aeruginosa.

Viruses causing infections treatable by methods of the present disclosure include, but are not limit to human papillomavirus, influenza, hepatitis A, B, C or D viruses, adenovirus, poxvirus, herpes simplex viruses, human cytomegalovirus, severe acute respiratory syndrome virus, ebola virus, measles virus, herpes virus (e.g., VZV, HSV-1, HAV-6, HSV-II, and CMV, Epstein Barr virus), flaviviruses, echovirus, rhinovirus, coxsackie virus, coronavirus, respiratory syncytial virus, mumpsvirus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumococci, meningococci and conococci, Klebsiella, Proteus, Serratia, Pseudomonas, Legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient in need thereof, they can be administered simultaneously, separately, sequentially, or in combination (e.g., for more than two agents).

### IV. Kits

The present disclosure also includes pharmaceutical kits useful, e.g., in the treatment or prevention of diseases or disorders associated with the activity of PD-L1 including its interaction with other proteins such as PD-1 and B7-1 (CD80), such as cancer or infections, which include one or more containers containing a pharmaceutical composition comprising a therapeutically effective amount of a pharmaceutical formulation comprising a compound of Formula (I), or a pharmaceutically acceptable salt thereof. Such kits can further include one or more of various conventional pharmaceutical kit components, such as, e.g., containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The following abbreviations may be used herein: aq. (aqueous); br (broad); d (doublet); dd (doublet of doublets); DCM (dichloromethane); DMF (N, N-dimethylformamide); Et (ethyl); EtOAc (ethyl acetate); g (gram(s)); h (hour(s)); HPLC (high performance liquid chromatography); Hz (hertz); J (coupling constant); LCMS (liquid chromatography-mass spectrometry); m (multiplet); M (molar); MS (Mass spectrometry); Me (methyl); MeCN (acetonitrile); MeOH (methanol); mg (milligram(s)); min. (minutes(s)); mL (milliliter(s)); mmol (millimole(s)); nM (nanomolar); NMR (nuclear magnetic resonance spectroscopy); Ph (phenyl); r.t. (room temperature), s (singlet); t (triplet or tertiary); TBS (tert-butyldimethylsilyl); tert (tertiary); tt (triplet of triplets); TFA (trifluoroacetic acid); THF (tetrahydrofuran); μg (microgram(s)); μL (microliter(s)); μM (micromolar); wt % (weight percent).

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The formulations of the Examples have been found to inhibit the activity of PD-1/PD-L1 protein/protein interaction according to at least one assay described herein.

### EXAMPLES

### Comparative Example A. Oral Pharmaceutical Formulation without Stabilizer

A 4800 g batch of the formulation in Table 1 was prepared according to the following procedure. The formulation was then compressed to form 50 mg tablets (240 mg total weight including excipients).

Compound 1 was ground using a mortar and pestle, and then screened through a 60 mesh screen. Next, the microcrystalline cellulose was added to the ground and screened Compound 1, and then blended for 12.5 minutes. Next, the sodium starch glycolate was added to the above blend, and blended for approximately 8.5 minutes. Finally, magnesium stearate was added to the above blend, and further blended for approximately 2.5 minutes. The final blend was then compressed into 240 mg tablet weight in a Korsch XL 100 tablet press using 8.5 mm round tooling.

TABLE 1

| | Percentage | |
|---|---|---|
| Component (Source) | (w/w) | mg/tablet |
| Compound 1 | 20.83 | 50.00 |
| Microcrystalline Cellulose (DuPont, | 74.37 | 178.48 |
| Avicel PH 102) | | |
| Sodium Starch Glycolate (JRS Pharma) | 4.00 | 9.60 |
| Magnesium Stearate (Mallinckrodt) | 0.80 | 1.92 |
| Total | 100.00 | 240.00 |

### Comparative Example B. Oral Pharmaceutical Formulation without Stabilizer

A 4800 g batch of the formulation in Table 2 was prepared according to the following procedure. The formulation was then compressed to form 100 mg tablets (480 mg total weight with excipients).

Compound 1 was ground using a mortar and pestle, and then screened through a 60 mesh screen. Next, the microcrystalline cellulose was added to the ground and screened Compound 1, and then blended for 12.5 minutes. Next, the sodium starch glycolate was added to the above blend, and blended for approximately 8.5 minutes. Finally, magnesium stearate was added to the above blend, and further blended for approximately 2.5 minutes. The final blend was then compressed into 480 mg tablet weight in a Korsch XL 100 tablet press using a 0.589×0.278 inch (14.96×7.06 mm) oval tooling.

TABLE 2

| | Percentage | |
|---|---|---|
| Component (Source) | (w/w) | mg/tablet |
| Compound 1 | 20.83 | 100.00 |
| Microcrystalline Cellulose (DuPont, | 74.37 | 356.96 |
| Avicel PH 102) | | |
| Sodium Starch Glycolate (JRS Pharma) | 4.00 | 19.2 |
| Magnesium Stearate (Mallinckrodt) | 0.80 | 3.84 |
| Total | 100.00 | 480.00 |

### Example 1. Oral Pharmaceutical Formulation with Stabilizer (400 mg of Compound 1)

A 600 g batch of the formulation in Table 3 was prepared according to the following procedure. The formulation was then compressed to form 400 mg tablets (750 mg total weight without the coating).

Compound 1 and fumaric acid were separately grinded and separately screened through a 60 mesh screen. The other excipients were screened through a 40 mesh screen separately. The grinded and screened Compound 1 and fumaric acid were then blended together for ten minutes. Next, the microcrystalline cellulose and mannitol were added to the blend of Compound 1 and fumaric acid, and then blended for 15 minutes. Next, the hydroxypropyl cellulose, sodium starch glycolate, and colloidal silicon dioxide were added to the above blend, and blended for ten minutes. Finally, magnesium stearate was added to the above blend, and further blended for three minutes. The final blend was then compressed into 400 mg tablets in a Piccola tablet press using a 0.689×0.328 inch (17.50×8.33 mm) oval tooling. The tablets were then coated with a 15% Opadry AMB II solution in water to a target 4% weight gain per tablet.

TABLE 3

| | | |
|---|---|---|
| | Percentage | |
| Component (Source) | (w/w) | mg/tablet |

Formulation (excluding coating)

| | | |
|---|---|---|
| Compound 1 | 53.33 | 400.00 |
| Microcrystalline Cellulose (DuPont, | 21.67 | 162.50 |
| Avicel PH 112)^{a} | | |
| Mannitol (Roguette, Pearlitol 200 SD)^{b} | 13.00 | 97.50 |
| Fumaric acid (Spectrum) | 2.50 | 18.75 |
| Hydroxypropyl cellulose (HPC) | 2.00 | 15.00 |
| (Ashland, Klucel EXF) | | |
| Sodium Starch Glycolate (JRS Pharma) | 6.00 | 45.00 |
| Colloidal Silicon Dioxide (Cabot/M-5P) | 0.50 | 3.75 |
| Magnesium Stearate (Spectrum) | 1.00 | 7.50 |
| Total | 100.00 | 750.00 |

Tablet Coating

| | |
|---|---|
| Film coating, Opadry AMB II | Target 4.0% |
| (88A530031 Orange) | weight gain |
| Solid concentration: 15.0% (w/w) | |

^{a}moisture content of not more than 1.5% by weight.
^{b}loss on drying of not more than 0.5% by weight.

### Example 2. Improved Stability of the Pharmaceutical Formulation of Example 1

### Analytical Methods

The High Performance Liquid Chromatography (HPLC) was conducted on an Agilent 1260 HPLC using Phenomenex Gemini NX-C18 PEEK column (3 μm, 4.6×150 mm, P/N 00F-4453-E0-BV) at column temperature of 20° C. (autosampler temperature 5° C.) with a mobile phase A of 0.1% NH₄OH in water and a mobile phase B of 0.1% NH₄OH in acetonitrile according to the gradient program in the table below. Injection volume of 5 μL, a detection wavelength of 254 nm, a flow rate of 1.0 mL/min, and a run time of 45 minutes were used. The relative retention times were measured relative to the retention time of Compound 1.

Gradient Program

| Time (min) | % mobile phase A | % mobile phase B |
|---|---|---|
| 0.0 | 85 | 15 |
| 2.0 | 85 | 15 |
| 18.0 | 70 | 30 |
| 21.0 | 70 | 30 |
| 25.0 | 60 | 40 |
| 36.0 | 5 | 95 |
| 39.0 | 5 | 95 |
| 39.5 | 85 | 15 |
| 45.0 | 85 | 15 |

### Stability Testing and Results

The stability of the tablets from Example 1 compared to those of Comparative Examples A and B were studied in an amber bottle with an oxygen absorbing packet (Example 1) or silica gel desiccant (Comparative Examples A and B). The tablets were analyzed by HPLC for the presence of degradants at time 0 (T0) and after 1 month (T1M), 3 months (T3M), and 6 months (T6M) at (a) 2-8° C. and (b) 25° C. and 60% relative humidity.

The presence of six major degradants (Compounds 2-7) were measured at each time point. Of these, the structures of Compounds 2-5 are shown in Table A. The structures of Compounds 6 and 7 are unknown. The relative retention times for Compounds 2-7 were approximately 0.76, 0.76, 0.84, 0.85, 1.17, and 1.18, respectively.

TABLE A

| Compound | Name and Structure |
|---|---|
| 2 | Diastereomers of (3R)-1-((8-((3′-(5-(((R)-3-carboxypyrrolidin-1-yl)methyl)-7- |
| 3 | cyanobenzo[d]oxazol-2-yl)- |
| | 2,2′-dimethyl-[1,1′-biphenyl]-3-yl)amino)-1,7-naphthyridin-3-yl)methyl)- |
| | 3-hydroxypyrrolidine 1-oxide* |
| | 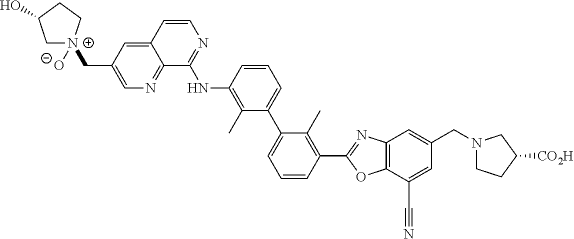 [SMILES: Cc1c(Nc2nccc3cc(C[N@+]4([O-])CC[C@@H](O)C4)cnc23)cccc1-c1cccc(-c2nc3cc(CN4CC[C@@H](C(=O)O)C4)cc(C#N)c3o2)c1C] |
| | 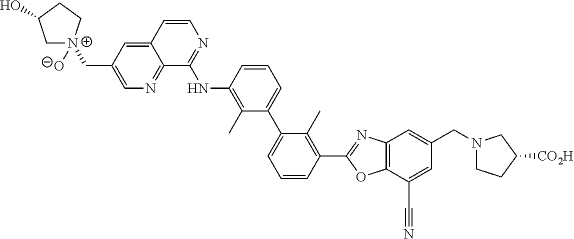 [SMILES: Cc1c(Nc2nccc3cc(C[N@@+]4([O-])CC[C@@H](O)C4)cnc23)cccc1-c1cccc(-c2nc3cc(CN4CC[C@@H](C(=O)O)C4)cc(C#N)c3o2)c1C] |
| 4 | Diastereomers of (3R)-3-carboxy-1-((7-cyano-2-(3′-((3-(((R)-3-hydroxypyrrolidin-1- |
| 5 | yl)methyl)-1,7-naphthyridin-8-yl)amino)-2,2′-dimethyl-[1,1′-biphenyl]-3-yl)benzo[d]oxazo |
| | 5-yl)methyl)pyrrolidine 1-oxide* |
| | 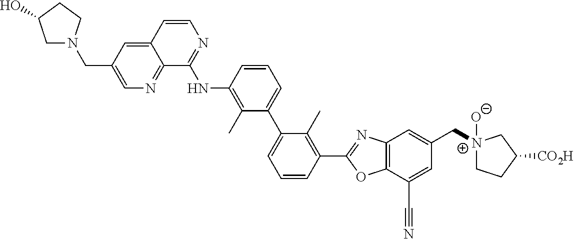 |
| | 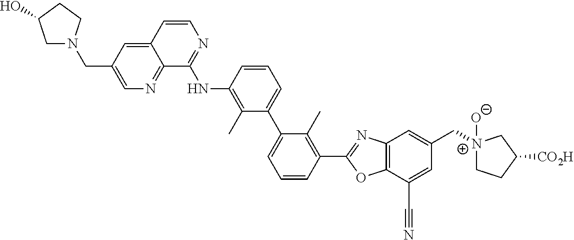 |

*Specific stereochemistry of the N-oxide has not been assigned for compounds 2-3 or 4-5

The % of each major degradant at each time point in the stability study for the tablets of Example 1, Comparative Example A, and Comparative Example B are shown in Tables 4, 5, and 6, respectively. Table 7 shows the relative 0% of each major degradant, as well as the total 0% of the six major degradants, after 6 months at (a) 2-8° C. and (b) 25° C. and 60% relative humidity. As shown in Table 7, the levels of degradants in the tablet of Example 1 after 6 months at 25° C. and 60% relative humidity are comparable to the levels of the degradants in the tablets of Comparative Examples A and B stored at refrigeration temperatures of 2-8° C. For example, the total % of degradants are in the tablet of Example 1 after 6 months at 25° C. and 60% relative humidity was 0.65%, while the total % of degradants in the tablets of Comparative Examples A and B stored at refrigeration temperatures of 2-8° C. was 0.65% and 0.66%, respectively. Further, the tablet of Example 1 shows improved stability with % total degradants that is about 38% and 42% of that of the tablets of Comparative Examples A and B after 6 months at (a) 2-8° C. and (b) 25° C. and 60% relative humidity, respectively. This data shows that the tablet of Example 1 solves the problem of eliminating the need for cold chain storage.

TABLE 4
(Tablet of Example 1)

| | | Primary degradants (%) | | | Primary degradants (%) | | |
|---|---|---|---|---|---|---|---|
| | | at 2-8° C. (Refrigerator) | | | at 25° C./60% RH | | |
| Degradant | T0 | T1M | T3M | T6M | T1M | T3M | T6M |
| Compound 2 | — | — | — | 0.09 | — | — | 0.12 |
| Compound 3 | 0.06 | 0.09 | 0.12 | — | 0.13 | 0.17 | 0.08 |
| Compound 4 | — | — | — | — | — | 0.06 | 0.07 |
| Compound 5 | — | — | 0.06 | 0.06 | 0.05 | 0.09 | 0.14 |
| Compound 6 | 0.05 | 0.09 | 0.08 | 0.10 | 0.08 | 0.13 | 0.18 |
| (structure unknown) | | | | | | | |
| Compound 7 | — | — | — | — | 0.05 | — | 0.06 |
| (structure unknown) | | | | | | | |

TABLE 5
(Tablet of Comparative Example A)

| | | Primary degradants (%) | | | Primary degradants (%) | | |
|---|---|---|---|---|---|---|---|
| | | at 2-8° C. (Refrigerator) | | | at 25° C./60% RH | | |
| Degradant | T0 | T1M | T3M | T6M | T1M | T3M | T6M |
| Compound 2 | — | — | — | 0.07 | — | 0.09 | 0.13 |
| Compound 3 | — | — | 0.05 | 0.09 | 0.05 | 0.13 | 0.19 |
| Compound 4 | — | — | 0.06 | 0.11 | 0.07 | 0.15 | 0.22 |
| Compound 5 | 0.08 | 0.14 | 0.15 | 0.24 | 0.21 | 0.41 | 0.55 |
| Compound 6 | — | — | — | 0.08 | 0.08 | 0.21 | 0.27 |
| (structure unknown) | | | | | | | |
| Compound 7 | — | — | 0.05 | 0.06 | 0.07 | 0.14 | 0.18 |
| (structure unknown) | | | | | | | |

TABLE 6
(Tablet of Comparative Example B)

| | | Primary degradants (%) | | | Primary degradants (%) | | |
|---|---|---|---|---|---|---|---|
| | | at 2-8° C. (Refrigerator) | | | at 25° C./60% RH | | |
| Degradant | T0 | T1M | T3M | T6M | T1M | T3M | T6M |
| Compound 2 | — | — | — | 0.07 | — | 0.06 | 0.13 |
| Compound 3 | — | — | — | 0.09 | — | 0.10 | 0.20 |
| Compound 4 | — | — | 0.06 | 0.12 | 0.07 | 0.12 | 0.23 |
| Compound 5 | 0.07 | 0.13 | 0.15 | 0.24 | 0.20 | 0.33 | 0.55 |
| Compound 6 | — | — | — | 0.07 | 0.08 | 0.17 | 0.26 |
| (structure unknown) | | | | | | | |
| Compound 7 | — | — | 0.05 | 0.07 | 0.07 | 0.12 | 0.17 |
| (structure unknown) | | | | | | | |

TABLE 7
(Comparison of Example 1 with Examples A and B)

| | Primary degradants (%) | | | Primary degradants (%) | | |
|---|---|---|---|---|---|---|
| | at 2-8° C. (Refrigerator) (T6M) | | | at 25° C./60% RH (T6M) | | |
| | | Comparative | Comparative | | Comparative | Comparative |
| Degradant | Example A | Example 1 | Example B | Example 1 | Example A | Example B |
| Compound 2 | 0.09 | 0.07 | 0.07 | 0.12 | 0.13 | 0.13 |
| Compound 3 | — | 0.09 | 0.09 | 0.08 | 0.19 | 0.20 |
| Compound 4 | — | 0.11 | 0.12 | 0.07 | 0.22 | 0.23 |
| Compound 5 | 0.06 | 0.24 | 0.24 | 0.14 | 0.55 | 0.55 |
| Compound 6 | 0.10 | 0.08 | 0.07 | 0.18 | 0.27 | 0.26 |
| Compound 7 | — | 0.06 | 0.07 | 0.06 | 0.18 | 0.17 |
| Total | 0.25 | 0.65 | 0.66 | 0.65 | 1.54 | 1.54 |

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

What is claimed is:

1. A pharmaceutical formulation, comprising a compound of Formula (I): 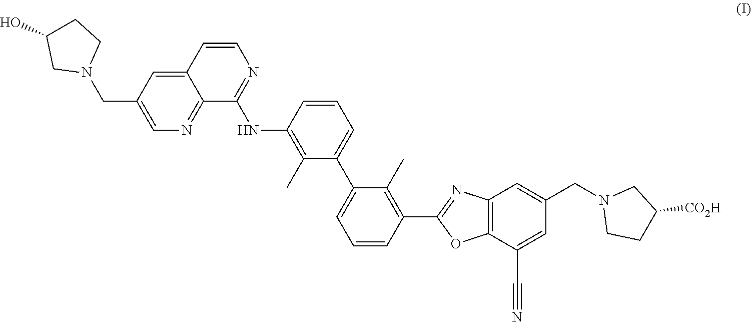 [SMILES: Cc1c(Nc2nccc3cc(CN4CC[C@@H](O)C4)cnc23)cccc1-c1cccc(-c2nc3cc(CN4CC[C@@H](C(=O)O)C4)cc(C#N)c3o2)c1C]
or a pharmaceutically acceptable salt thereof, and a stabilizing agent.

2. The pharmaceutical formulation of claim 1, wherein the compound of Formula (I), or the pharmaceutically acceptable salt thereof, is present as a free base.

3. The pharmaceutical formulation of claim 1, wherein the compound of Formula (I), or the pharmaceutically acceptable salt thereof, is present in an amount on a free base basis of from about 40% to about 80% by weight of the formulation.

4. The pharmaceutical formulation of claim 1, wherein the compound of Formula (I), or the pharmaceutically acceptable salt thereof, is present in an amount on a free base basis of from about 40% to about 70% by weight of the formulation.

5. The pharmaceutical formulation of claim 1, wherein the stabilizing agent is an antioxidant.

6. The pharmaceutical formulation of claim 1, wherein the stabilizing agent is an organic acid or an ester thereof, a metal sulfite, a metal bisulfite, a metal metabisulfite, a metal thiosulfate, a metal formaldehyde sulfoxylate, or an alkylated phenol, or a mixture of any of the aforementioned.

7. The pharmaceutical formulation of claim 1, wherein the stabilizing agent is ascorbic acid, fumaric acid, citric acid, tartaric acid, ascorbyl palmitate, propyl gallate, sodium sulfite, sodium bisulfite, sodium metabisulfite, sodium thiosulfate, sodium formaldehyde sulfoxylate, butylated hydroxyanisole, butylated hydroxytoluene, cysteine, or tocopherol.

8. The pharmaceutical formulation of claim 1, wherein the stabilizing agent is present in an amount of from about 0.001% to about 20% by weight of the formulation.

9. The pharmaceutical formulation of claim 1, wherein the stabilizing agent is present in an amount of from about 0.001% to about 10% by weight of the formulation.

10. The pharmaceutical formulation of claim 1, wherein the stabilizing agent is present in an amount of from about 0.001% to about 1% by weight of the formulation.

11. The pharmaceutical formulation of claim 1, wherein the stabilizing agent is an organic acid.

12. The pharmaceutical formulation of claim 11, wherein the organic acid is C₁₋₈ alkyl carboxylic acid, which is optionally substituted by 1, 2, 3, 4, 5, or 6 substituents independently selected from SH, NH₂, OH, and CO₂H.

13. The pharmaceutical formulation of claim 11, wherein the organic acid is C₂₋₈ alkenyl carboxylic acid, which is optionally substituted by 1, 2, 3, 4, 5, or 6 substituents independently selected from SH, NH₂, OH, and CO₂H.

14. The pharmaceutical formulation of claim 11, wherein the organic acid is fumaric acid, citric acid, succinic acid, adipic acid, maleic acid, sorbic acid, malonic acid, glutaric acid, gluconic acid, lactic acid, glycolic acid, malic acid, tartaric acid, tartronic acid, galactaric acid, glutamic acid, aspartic acid, benzoic acid, phthalic acid, isophthalic acid, terephthalic acid, or trimelitic acid.

15. The pharmaceutical formulation of claim 11, wherein the organic acid is fumaric acid, citric acid, tartaric acid, succinic acid, adipic acid, or maleic acid.

16. The pharmaceutical formulation of claim 11, wherein the organic acid is fumaric acid.

17. The pharmaceutical formulation of claim 11, wherein the organic acid is present in an amount of from about 0.5% to about 20% by weight of the formulation.

18. The pharmaceutical formulation of claim 11, wherein the organic acid is present in an amount of from about 1% to about 5% by weight of the formulation.

19. The pharmaceutical formulation of claim 11, wherein the organic acid is present in an amount of from about 1% to about 4% by weight of the formulation.

20. The pharmaceutical formulation of claim 11, wherein the organic acid is an antioxidant.

21. The pharmaceutical formulation of claim 1, wherein the pharmaceutical formulation further comprises one or more excipients.

22. The pharmaceutical formulation of claim 21, wherein the one or more excipients are present in an amount of from about 5% to about 90% by weight of the formulation.

23. The pharmaceutical formulation of claim 21, wherein the one or more excipients are present in an amount of from about 5% to about 70% by weight of the formulation.

24. The pharmaceutical formulation of claim 21, wherein the one or more excipients are present in an amount of from about 20% to about 60% by weight of the formulation.

25. The pharmaceutical formulation of claim 21, wherein the one or more excipients is microcrystalline cellulose, silicified microcrystalline cellulose, mannitol, lactose, sucrose, dextrose, sorbitol, xylitol, starch, sodium starch, calcium phosphate, an alginate, calcium carbonate, sodium carbonate, sodium chloride, calcium sulphate, calcium lactate, sodium chloride, a wax, a clay, talc, tragacanth, glucose, acacia, guar gum, agar, povidone, crospovidone, copovidone, poly(vinyl pyrrolidone-co-vinyl acetate), gelatin, hydroxypropyl cellulose, hydroxypropyl methyl cellulose, hydroxypropyl methylcellulose acetate stearate, methyl cellulose, ethyl cellulose, cellulose, silicified cellulose, carboxymethylcellulose, croscarmellose sodium, carboxymethylcellulose calcium, sodium starch glycolate, an ion-exchange resin, or a mixture of any of the aforementioned.

26. The pharmaceutical formulation of claim 21, wherein the one or more excipients comprises at least one excipient having a moisture content of not more than about 2% by weight of the excipient.

27. The pharmaceutical formulation of claim 21, wherein the one or more excipients comprises at least one excipient having a moisture content of not more than about 1.5% by weight of the excipient.

28. The pharmaceutical formulation of claim 21, wherein the one or more excipients, having a moisture content of not more than about 2% or not more than about 1.5% by weight of the excipient, comprises at least 15% by weight of the formulation.

29. The pharmaceutical formulation of claim 21, wherein the one or more excipients, having a moisture content of not more than about 2% or not more than about 1.5% by weight of the excipient, comprises at least 20% by weight of the formulation.

30. The pharmaceutical formulation of claim 1, wherein the pharmaceutical formulation further comprises a glidant component or lubricant component.

31. The pharmaceutical formulation of claim 30, wherein the glidant component or lubricant component comprises magnesium stearate, silicon dioxide, sodium stearyl fumarate, calcium stearate, stearic acid, a hydrogenated oil, polyethylene glycol, a starch, an alginate, glyceryl monostearate, glyceryl behenate, glyceryl palmitostearate, a fatty acid, a poloxamer, a metal stearate, a metal fatty acid salt, talc, a clay, or a silicate, or a mixture of any of the aforementioned.

32. The pharmaceutical formulation of claim 1, further comprising a diluent component.

33. The pharmaceutical formulation of claim 32, wherein the diluent component is present in an amount of from about 5% to about 90% by weight of the formulation.

34. The pharmaceutical formulation of claim 32, wherein the diluent component is present in an amount of from about 20% to about 50% by weight of the formulation.

35. The pharmaceutical formulation of claim 32, wherein the diluent component comprises microcrystalline cellulose, mannitol, lactose, sucrose, dextrose, starch, sorbitol, dibasic calcium phosphate, cellulose, hydroxypropyl cellulose, xylitol, sodium carbonate, calcium phosphate, hydroxypropyl methylcellulose, hydroxypropyl methylcellulose acetate stearate, calcium sulfate, calcium lactate, calcium carbonate, sodium chloride, povidone, or a clay, or a mixture of any of the aforementioned.

36. The pharmaceutical formulation of claim 32, wherein the diluent component comprises microcrystalline cellulose and mannitol.

37. The pharmaceutical formulation of claim 36, wherein the ratio of microcrystalline cellulose to mannitol is about 1:1 to about 2:1.

38. The pharmaceutical formulation of claim 32, wherein the diluent component comprises at least one diluent having a moisture content of not more than about 2% by weight of the diluent.

39. The pharmaceutical formulation of claim 32, wherein the diluent component comprises at least one diluent having a moisture content of not more than about 1.5% by weight of the diluent.

40. The pharmaceutical formulation of claim 1, further comprising a disintegrant component.

41. The pharmaceutical formulation of claim 40, wherein the disintegrant component is present in an amount of from about 2% to about 10% by weight of the formulation.

42. The pharmaceutical formulation of claim 40, wherein the disintegrant component comprises sodium starch glycolate, croscarmellose sodium, povidone, crospovidone, pregelatinized starch, starch, guar gum, an alginate, an ion-exchange resin, a clay, talc, methyl cellulose, ethyl cellulose, calcium carbonate, carboxymethylcellulose calcium, carboxymethyl cellulose sodium, or agar, or a mixture of any of the aforementioned.

43. The pharmaceutical formulation of claim 40, wherein the disintegrant component comprises sodium starch glycolate.

44. The pharmaceutical formulation of claim 1, further comprising a glidant component.

45. The pharmaceutical formulation of claim 44, wherein the glidant component is present in an amount of from about 0.01% to about 5% by weight of the formulation.

46. The pharmaceutical formulation of claim 44, wherein the glidant component is present in an amount of from about 0.1% to about 2% by weight of the formulation.

47. The pharmaceutical formulation of claim 44, wherein the glidant component comprises silicon dioxide, talc, starch, a silicate, a clay, or a mixture of any of the aforementioned.

48. The pharmaceutical formulation of claim 44, wherein the glidant component comprises colloidal silicon dioxide.

49. The pharmaceutical formulation of claim 1, further comprising a lubricant component.

50. The pharmaceutical formulation of claim 49, wherein the lubricant component is present in an amount of from about 0.01% to about 5% by weight of the formulation.

51. The pharmaceutical formulation of claim 49, wherein the lubricant component is present in an amount of from about 0.5% to about 2% by weight of the formulation.

52. The pharmaceutical formulation of claim 49, the lubricant component comprises magnesium stearate, sodium stearyl fumarate, stearic acid, a hydrogenated oil, an alginate, polyethylene glycol, glyceryl monostearate, glyceryl palmitostearate, glyceryl behenate, calcium stearate, talc, starch, a metal lauryl sulfate, mineral oil, a fatty acid, a poloxamer, or a metal stearate, or a mixture of any of the aforementioned.

53. The pharmaceutical formulation of claim 49, wherein the lubricant component comprises magnesium stearate.

54. The pharmaceutical formulation of claim 1, further comprising a binder component.

55. The pharmaceutical formulation of claim 54, wherein the binder component is present in an amount of from about 0.1% to about 20% by weight of the formulation.

56. The pharmaceutical formulation of claim 54, wherein the binder component is present in an amount of from about 0.1% to about 5% by weight of the formulation.

57. The pharmaceutical formulation of claim 54, the binder component comprises hydroxypropyl cellulose, hydroxypropyl methylcellulose, povidone, copovidone, poly(vinyl pyrrolidone-co-vinyl acetate), tragacanth, acacia, starch, sodium starch, methyl cellulose, ethyl cellulose, gelatin, glucose, carboxymethylcellulose calcium, or carboxymethylcellulose sodium, or a mixture of any of the aforementioned, or a mixture of any of the aforementioned.

58. The pharmaceutical formulation of claim 54, wherein the binder component comprises hydroxypropyl cellulose.

59. The pharmaceutical formulation of claim 1, wherein the six month stability of the pharmaceutical formulation at a temperature of about 25° C. and about 60% relative humidity is comparable to the six month stability of a formulation comprising a compound of Formula (I), or a pharmaceutically acceptable salt thereof, without the stabilizing agent at a refrigeration temperature.

60. The pharmaceutical formulation of claim 1, wherein the total amount (%) of major degradants of the pharmaceutical formulation after storage at a temperature of about 25° C. and about 60% relative humidity for six months is comparable to the total amount (%) of major degradants of a formulation comprising a compound of Formula (I), or a pharmaceutically acceptable salt thereof, without the stabilizing agent after storage at a refrigeration temperature for six months.

61. The pharmaceutical formulation of claim 1, wherein the pharmaceutical formulation does not require cold chain storage.

62. An oral dosage form comprising the pharmaceutical formulation of claim 1.

63. The oral dosage form of claim 62, wherein the compound or the salt is present in an amount of about 400 mg to about 800 mg.

64. The oral dosage form of claim 62, wherein the compound or the salt is present in an amount of about 400 mg.

65. The oral dosage form of claim 62, which is a tablet.

66. The oral dosage form of claim 62, which is a capsule.

67. The oral dosage form of claim 62, further comprising an outer coating.

68. A method of inhibiting PD-1/PD-L1 interaction, comprising administering to a patient in need thereof the oral dosage form of claim 62.

69. A method of reducing the amount of cell surface PD-L1, comprising administering to a patient in need thereof the oral dosage form of claim 62.

70. A method of decreasing or reducing the interaction of PD-1 and PD-L1, comprising administering to a patient in need thereof the oral dosage form of claim 62.

71. A method of enhancing, stimulating and/or increasing an immune response in a patient in need thereof, comprising administering to the patient in need thereof the oral dosage form of claim 62.

72. The method of claim 71, wherein the immune response is a T cell immune response.

73. The method of claim 72, wherein the T cell immune response is a cytotoxic or effector T cell response.

74. A method of treating a PD-1-related disease or condition, comprising administering to a patient in need thereof the oral dosage form of claim 62.

75. The method of claim 74, wherein the disease or condition is an infection disease, inflammation, autoimmune disease, cancer, or neurodegenerative disorder.
